(19) United States
(12) Patent Application Publication
(10) Pub. No.: US 2007/0071677 A1
(43) Pub. Date: Mar. 29, 2007

(54) Non-toxic membrane-translocating peptides

(76) Inventors: Yoon Jeong Park, Seoul (KR); Jun Feng Liang, Westfield, NJ (US); Victor C. Yang, Ann Arbor, MI (US); Li-Chien Chang, Taipei (TW)

Correspondence Address: MAYER, BROWN, ROWE & MAW LLP, 1909 K STREET, N.W., WASHINGTON, DC, 20006, US

(21) Appl. No.: 10/548,438

(22) PCT Filed: Mar. 10, 2004
(86) PCT No.: PCT/US04/07145

§ 371 Date: Sep. 14, 2006

(30) Foreign Application Priority Data
Mar. 10, 2003 (US) 60452929

(51) Int. Cl. A61K 51/00; A61K 38/55; A61K 49/00; A61K 39/395; A61K 48/00; A61K 38/10; A61K 38/08

(52) U.S. Cl. 424/1.69; 514/7; 514/15; 514/16; 514/17; 424/178.1; 424/9.6

(57) ABSTRACT

Compositions for transport across a biological membrane include a membrane-translocating LMWP peptide and a cargo molecule. Methods for transporting a cargo molecule across a biological membrane are also described.

### PRIORITY CLAIM

This application claims priority to U.S. Provisional Application No. 60/452,929, filed Mar. 10, 2003, which is incorporated herein in its entirety.

### GRANT STATEMENT

This work was supported by grant numbers R01HL38353, R44HL49705, R01HL55461, and R01GM068942 from the United States National Institutes of Health, and by NASA SBIR grant numbers R43 HL59705 and R44HL59705. Thus, the U.S. Government may have certain rights in the invention.

### FIELD OF THE INVENTION

The present invention generally relates to the field of cell transduction and cell transformation. More particularly, the present invention relates to membrane-translocating non-toxic peptides and conjugates, and methods for using the same.

Table of Abbreviations

| | |
|---|---|
| 293 cells | human embryonic kidney cells |
| CT26 cells | murine adenocarcinoma colon cancer cells |
| DMEM | Dulbecco's modified essential medium |
| FACS | fluorescent-activated cell sorting |
| FBS | fetal bovine serum |
| FITC | fluoroisothiocyanate |
| HeLa cells | human epithelial cells |
| HIV | human immunodeficiency virus |
| HPLC | high performance liquid chromatography |
| IC₅₀ | concentration producing 50% inhibition |
| LMWP | low molecular weight protamine |
| MALDI-MS | matrix-assisted laser desorption/ionization mass |
| | spectrometry |
| MALDI- | matrix-assisted laser desorption/ionization time-of-flight |
| TOF-MS | mass spectrometry |
| MCF-7 cells | human breast cancer cells |
| MWCO | molecular weight cutoff |
| OD | optical density |
| ONPG assay | colorimetric β-galactosidase enzyme activity assay |
| PAGE | polyacrylamide gel electrophoresis |
| PBS | phosphate-buffered saline |
| pDNA | plasmid DNA |
| PEI | polyethylene imine |
| PTD | protein transduction domain |
| SDS | sodium dodecyl sulfate |
| TAT | HIV transactivator protein |
| TDSP | thermolysin-digested segmented protamine |

### BACKGROUND OF THE INVENTION

The potential for intracellular imaging and therapeutic use of proteins, peptides, and oligonucleotides has been limited by the impermeable nature of the cell membrane to these compounds. Efficient delivery of therapeutic and imaging compounds is typically achieved only when such agents are hydrophobic and small; typically less than 600 Daltons (Schwarze et al. (1999) Science 285: 1569-72). The most effective means to date for intracellular delivery of biomolecules has been by the receptor- or transporter-mediated endocytosis process. This method, however, suffers from a low efficiency and, above all, is not quite suitable for delivering hydrophilic macromolecules such as therapeutic proteins and nucleic acids.

Recently, several small regions of proteins termed protein transduction domains (PTDs) including peptides of the human immunodeficiency virus (HIV) TAT protein (Fawell et al. (1994) Proc Natl Acad Sci USA 91: 664-8), the Drosophila homeotic transcription factor ANTP2, and the herpes simplex virus type 1 (HSV-1) VP223, have received significant and widespread attention within the pharmaceutical and medical societies, due to their unprecedented ability to deliver such macromolecules into living cells. By covalently linking these PTDs to a variety of species including hydrophilic fluorescent probes (Vives et al. (1997) J Biol Chem 272: 16010-7), macromolecular proteins (Schwarze et al. (1999) Science 285: 1569-72; Fawell et al. (1994) Proc Natl Acad Sci USA 91: 664-8), and nano-carriers such as magnetic nano-particles (Josephson et al. (1999) Bioconjug Chem 10: 186-91) and liposomes (Torchilin et al. (2001) Proc Natl Acad Sci USA 98: 8786-91), these peptides were shown to be capable of translocating all such attached species into all cell types both in vitro and in vivo.

Cell-internalization by PTDs is highly efficient and occurs without perturbing or damaging cellular membranes. In addition, since this PTD-mediated membrane transduction was demonstrated to occur in a receptor- and transporter-independent fashion, all cell types are believed to be transducible. See Schwarze et al. (1999) Science 285: 1569-72; Suzuki et al. (2002) J Biol Chem 277: 2437-43; Niesner et al. (2002) Bioconjug Chem 13: 729-36.

Despite the potential of PTDs as universal carriers for intracellular delivery of biomolecules, the clinical use of PTDs has been hindered by two major drawbacks. First, all available PTDs are derived from highly infective viral proteins, and the toxicity and immunogenicity of these peptides has not been established. Second, synthesis of these PTDs is expensive, time-consuming, and of a low yield unsuitable for numerous clinical applications.

Thus, there exists a long-standing need in the art for cell transformation and drug delivery methods having improved efficiency and safety as well as reduced cost. In particular, there exists a need for identification of PTDs from nontoxic and nonvirulent sources, and reliable, economically feasible methods for large scale production of such PTDs. To meet this need, the present invention provides nontoxic membrane-translocating peptides derived from low molecular weight protamine (LMWP). The present invention also provides high yield methods for preparing the LMWP peptides, and methods for using the same.

### SUMMARY OF THE INVENTION

The present invention provides non-toxic membrane-translocating peptides and compositions. In a representative embodiment of the invention, a non-toxic composition for transport across a biological membrane comprises a membrane-translocating LMWP peptide and a cargo molecule, wherein the LMWP peptide is conjugated to, complexed with, fused to, or otherwise associated with the cargo molecule. Also provided are pharmaceutical compositions comprising a membrane-translocating LMWP peptide, a drug, and a pharmaceutically acceptable carrier.

A membrane-translocating LMWP peptide is preferably a purified thermolysin-digested protamine peptide. Representative membrane-translocating LMWP peptides are set forth as SEQ ID NOs: 1-4.

In accordance with the present disclosure, cargo molecules and drugs each include, but are not limited to, therapeutic agents, diagnostic agents, binding agents, heterologous agents, and combinations thereof. In a representative embodiment of the invention, a composition for transport across a biological membrane is prepared using a cytotoxic therapeutic agent. More specifically, a cytotoxin can comprise a protein synthesis inhibitor, such as gelonin. In another representative embodiment of the invention, a composition for transport across a biological membrane comprises a diagnostic agent, such as a detectable label selected from the group consisting of a radionuclide, a metal ion, gas microbubbles, a fluorophore, an epitope, and a radioactive label.

Cargo molecules and drugs used in accordance with the present invention can each also include, but are not limited to, peptides, polypeptides, polymeric conjugates (e.g., polymers conjugated to antibiotics), nucleic acids, small molecules, antibodies, peptide nucleic acids, carbohydrates, vitamins, hormones, odorants, pheromones, toxins, and combinations thereof. In representative embodiments of the invention, a protein (e.g., gelonin) is used as the cargo molecule or drug. In other representative embodiments of the invention, a nucleic acid (e.g., a plasmid) is used as the cargo molecule or drug. Nucleic acids can be directly complexed with membrane-translocating LMWP peptides. The nucleic acids are also condensed when complexed with the LMWP peptides, which reduced size facilitates membrane translocation.

The present invention further provides methods for transporting or enhancing the transport of a cargo molecule across a biological membrane. In a representative embodiment of the invention, the method comprises contacting a biological membrane with a composition comprising a membrane-translocating LMWP peptide and a cargo molecule, whereby the cargo molecule is transported across a biological membrane. A biological membrane can comprise a cellular membrane, including the cell membrane of a prokaryotic cell (e.g., a bacterial cell) or a eukaryotic cell (e.g., a human cell), or an intracellular membrane, such as a nuclear membrane. To perform the transport methods of the invention, a biological membrane can exist in vitro, ex vivo, or in vivo.

The present invention further provides methods for drug delivery to a subject, the method comprising administering to a subject a composition for transport across a biological membrane, wherein the composition comprises a membrane-translocating LMWP peptide, a drug, and a pharmaceutically acceptable carrier; and whereby the drug is delivered to cells of the subject. The drug delivery methods are appropriate for use in mammalian subjects, including human subjects.

### BRIEF DESCRIPTION OF THE DRAWINGS

FIGS. 1A-1B depict cell translocation activity of LMWP peptides.

FIGS. 1A-1D are photographs of FITC-labeled TDSP5 and FITC-labeled TAT following cellular uptake. FIG. 1A, FITC-labeled TDSP5 incubated with cells for 15 minutes; FIG. 1B, FITC-labeled TDSP5 incubated with cells for 1 hour; FIG. 1C, FITC-labeled TAT incubated with cells for 15 minutes; FIG. 1D, FITC-labeled TAT incubated with cells for 1 hour.

FIG. 2 is a line graph depicting the time course of cellular uptake of labeled LMWP (●) and TAT (▪) peptides. The time course was measured by FACS analysis. The cellular uptake of each peptide was estimated based on the mean fluorescent signal of 10,000 cells collected.

FIGS. 3A-3B show cellular uptake of LMWP peptides.

FIG. 3A is a bar graph that shows percentage uptake of FITC-labeled LMWP peptides into cells, which was performed as described in Example 3. FITC-labeled LMWPs were applied onto each group of cells grown in the presence of 10% serum for 30 minutes at 37° C. Cell uptake was determined by counting fluorescence with FACS analysis. Stippled bar, 293 cells; cross-hatched bar, HeLa cells; gray bar, CT26 cells; black bar, MCF-7 cells.

FIG. 3B shows a FACS analysis of uptake of LMWPs by 293 cells, performed as described in Example 3.

FIGS. 4A-4C show cellular uptake of TDSP5 in response to varying temperature and culture conditions.

FIG. 4A shows a FACS analysis of uptake of FITC-labeled TDSP5 by 293 cells, which was performed as described in Example 3. TDSP5 uptake was similar at 4° C. and at 37° C.

FIG. 4B shows photographs of MCF-7 human breast cancer cells cultured in the presence of FITC-labeled TDSP5 at 37° C. (left panel) and at 4° C. (right panel). The FITC label appears TDSP5 uptake was similar at 4° C. and at 37° C.

FIG. 4C is a bar graph that shows uptake of FITC-labeled TDSP5 by cells cultured in the presence or absence of FBS. Cellular uptake of TDSP5 was assayed by FACS analysis as described in Example 3. TDSP5 uptake was similar in the absence (0%) FBS or in 10% FBS. Stippled bars, 293T cells; cross-hatched bars, HeLa cells; gray bars, CT26 cells; black bars, MCF-7 cells.

FIG. 5 is a bar graph showing the results of cell cytotoxicity assays, which were performed as described in Example 4. The LMWP peptides showed little or no toxicity. TAT peptide showed significant cytotoxicity when applied to cells at concentrations of 5.0 mM and 10.0 mM. Stippled bars, TDSP2; diagonal cross-hatched bars, TDSP3; gray bars, TDSP4; black bars, TDSP5; horizontal cross-hatched bars, TAT.

FIGS. 6A-6C depict steps in the formation of LMWP-gelonin conjugates, which were prepared as described in Example 5.

FIG. 6A shows the elution profile (280 nm) following heparin affinity chromatography.

FIG. 6B is a photograph of a SDS-PAGE gel. Native gelonin and LMWP-gelonin samples were stained with Coomassie Brilliant Blue.

FIG. 6C is a MALDI-MS profile of LMWP-gelonin conjugates. Molecular weight was determined using matrix assisted laser desorption-time of flight mass spectrometric analysis.

FIG. 7A depicts results of FACS analysis following cell uptake of rhodamine-labeled LMWP-gelonin conjugate and rhodamine-labeled TAT-gelonin conjugate. The uptake assays were performed using CT-26 cells (1×10⁶ cells/well) in the presence of 10% serum, as described in Example 6. LMWP-gelonin conjugate and TAT-gelonin conjugate showed similar uptake levels.

FIGS. 7B-7C depict tumor penetration of rhodamine-labeled LMWP-gelonin conjugate (FIG. 7B) and rhodamine-labeled free gelonin (FIG. 7C) in a mouse model of colon cancer. The rhodamine label appears as darker areas in the photograph. The extent of penetration of TAT-gelonin conjugate was also analyzed. Tumors were isolated at 10 hours after injection of either rhodamine-labeled gelonin-TAT conjugate or rhodamine-labeled gelonin, sectioned, and imaged.

FIG. 8A is a line graph that depicts survival of CT-26 cells following incubation with native gelonin (▪); TAT-Gelonin (●); and LMWP-Gelonin (▴). The indicated doses of each compound were added to the 96-well culture plates containing approximately 5000 cells/well. The plates were incubated for 48 hours at 37° C. under an atmosphere of 5% CO₂ in assay. The number of remaining cells was determined using an MTT assay and then compared to those of untreated cells in the control wells, as described in Example 7. Values represent means±standard deviation. Each experiment was performed in triplicate.

FIG. 8B is a photograph of excised tumors from treated mice, which shows the antitumor effects of LMWP-gelonin. Mice received the indicated treatments as described in Example 7. The average tumor masses of treated mice were as follows: PBS solution (3.16±0.65 g, N=5); 100 μg of gelonin (2.62±0.53 g; N=4); 110 μg of LMWP-gelonin (0.33±0.12 g; N=5); 10 μg of LMWP and 100 μg of gelonin mixture (2.74±0.68 g; N=4).

FIG. 9 is a photograph showing results of a DNase protection assay, performed as described in Example 5. The complex solution was incubated with 50 units of DNase I for 10, 20, 40, 60, and 80 minutes. After incubation, DNA was analyzed by 1% agarose gel electrophoresis.

FIGS. 10A-10C depict features of the pDNA/LMWP(TDSP5) complex.

FIG. 10A is a photograph of showing the results of a gel retardation assay. The pDNA/LMWP complexes were prepared at the indicated charge ratios and incubated at room temperature for 20 minutes to allow complex formation. The complexes were analyzed by 1% (w/v) agarose gel electrophoresis.

FIG. 10B is a line graph showing particle size of pDNA/LMWP(TDSP5) complexes as a function of the charge ratio (+/−) between LMWP and plasmid DNA. Data are presented as mean±standard deviation. The plasmid DNA concentration was 2 μg/ml.

FIG. 10C is a line graph showing Zeta potential of pDNA/LMWP(TDSP5) complexes as a function of the charge ratio (+/−) between LMWP and plasmid DNA. Data were presented as mean±standard deviation. The plasmid DNA concentration was 2 μg/ml.

FIGS. 11A-11B depict the results of FACS analysis following uptake of FITC-labeled DNA/peptide complexes into cells.

FIG. 11A depicts flow cytometry analysis of FITC-labeled pDNA/LMWP(TDSP5) complexes in cells. DNA entry into cells depended on formation of a complex with LMWP. The concentration of pDNA was 5 μg/ml, and the incubation time at 37° C. was 1 hour.

FIG. 11B depicts flow cytometry analysis of FITC-labeled pDNA/TAT complexes in cells. DNA entry into cells depended on formation of a complex with TAT. The concentration of pDNA was 5 μg/ml, and the incubation time at 37° C. was 1 hour.

FIGS. 12A-12D are bar graphs depicting transfection efficiency of the pDNA/LMWP(TDSP5) complex into 293T cells. Transfection efficiency was measured using a colorimetric β-galactosidase enzyme activity assay (ONPG assay) as described in Example 9.

FIG. 12A is a bar graph showing the effects of plasmid DNA content on transfection efficiency. The charge ratio of the pDNA/LMWP(TDSP5) was adjusted to (−/+) 1:2.

FIG. 12B is a bar graph showing the effects of charge ratio (−/+) on pDNA/LMWP(TDSP5) transfection efficiency. Plasmid DNA content was adjusted to 5 μg.

FIG. 12C is a bar graph comparing the transfection efficiency of pDNA/LMWP(TDSP5) versus pDNA/TAT at a charge ratio of 1:5 (−/+).

FIG. 12D is a bar graph comparing the transfection efficiency of pDNA/LMWP(TDSP5) and various charge ratios (−/+) of pDNA/PEI complexes. PEI having a similar molecular weight (2000 Da) to that of LMWP (1880 Da) was used for comparison. The data were expressed as mean±standard deviation of four experiments. Asterisk (*), p<0.05, as compared with that of plasmid DNA and that of PEI complex at a same charge ratio.

FIG. 13 is a bar graph depicting cytotoxicity of 293T cells when exposed to LMWP or PEI. Cells were plated on 96 wells and exposed to LMWP, pDNA/LMWP(TDSP5) complex, PEI, or pDNA/PEI complex. Cytotoxicity tests were conducted using a MTT colorimetric assay as described in the Example 10. The data were expressed as mean±standard deviation of four experiments. Asterisk (*), p<0.05, as compared with that of PEI or PEI complex; double asterisk (**), p<0.05, compared with that with control.

### DETAILED DESCRIPTION OF THE INVENTION

### I. Definitions

While the following terms are believed to be well understood by one of ordinary skill in the art, the following definitions are set forth to facilitate explanation of the invention.

The terms “translocate” and “transduce,” and variations thereof, are used interchangeably herein to refer to the activity of a peptide, peptide conjugate, or peptide complex in traversing a biological membrane. Where the biological membrane is a cell membrane, the process of translocating permits entry of the peptide in to a cell by a process other than receptor mediated endocytosis.

The term “biological membrane” refers to a cellular or intracellular lipid-containing barrier. Representative biological membranes include but are not limited to nuclear membranes, endosomal membranes, endoplasmic reticulum membranes, lysosomal membranes, organelle membranes, etc. The term “biological membrane” also encompasses ex vivo membranes.

The term “membrane-translocating peptide” refers to a peptide that is capable of traversing a cellular membrane to thereby enter a cell. A “membrane-translocating peptide” also preferably mediates the translocation of a cargo molecule.

The description “composition for transport across a biological membrane” refers to a composition comprising a membrane-translocating LMWP peptide, and a cargo molecule or drug, wherein the LMWP peptide is conjugated with, complexed with, fused to the LMWP peptide, or otherwise associated with a LMWP peptide. In this context, the term “associated with” refers to a physical or otherwise linking association, and excludes a simple admixture.

The term “protamine” refers to a polycationic peptide, which is typically derived from salmon sperm. Protamine is alternatively known as “salmine protamine” or “n-protamine.” See Ando et al. (1973) in Protamine: Molecular Biology, Biochemistry and Biophysics, ed. Kleinzeller, Springer-Verlag, New York, Vol. 12, pp. 1-109 and U.S. Pat. Nos. 5,919,761 and 5,534,619.

The term “low molecular weight protamine,” abbreviated as “LMWP,” refers to protamine fragments produced by enzymatic digestion of protamine using thermolysin, which can be prepared as described in Example 1. The terms “low molecular weight protamine” and “LMWP” also include peptides having an amino acid sequence of protamine fragments produced by enzymatic digestion of protamine using thermolysin. Representative LMWP peptides are set forth as SEQ ID NOs: 1-4.

The terms “cargo” and “cargo molecule” are used herein interchangeably to refer to a delivery substrate, i.e. any molecule intended to be delivered into a cell. A cargo molecule can be derived from any source and thus includes naturally occurring, synthetic, and recombinantly produced molecules. A cargo can be a purified molecule, a homogenous sample, or a mixture of molecules or compounds. Functionally, a cargo molecule can comprise a therapeutic agent, a diagnostic agent, a binding agent, a heterologous agent, and combinations thereof. Representative cargo molecules include but are not limited to peptides, polypeptides, polymeric conjugates, nucleic acids, small molecules, antibodies, peptide nucleic acids, carbohydrates, vitamins or derivative thereof, hormones, odorants, pheromones, and toxins.

The terms “nucleic acid molecule” and “nucleic acid” each refer to deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. Representative nucleic acids include plasmids, genes, cDNAs, RNAs (e.g., antisense RNAs and double-stranded RNAs), and aptamers.

The terms “peptide” and “polypeptide” and “protein” each refer to a compound made up of a single chain of D- or L-amino acids or a mixture of D- and L-amino acids joined by peptide bonds. Generally, peptides contain at least two amino acid residues and are less than about 50 amino acids in length. Polypeptides and proteins are generally greater than 50 amino acids in length and may have substantial three-dimensional structure.

The term “peptide nucleic acid” refers to a nucleic acid analogue in which the backbone is a neutral pseudopeptide rather than a sugar.

The term “polymeric conjugate” refers to a biocompatible biomedical polymers (e.g., chitosan, polylactide) in which monomers in the backbone are conjugated to small drugs (e.g., doxorubicin, tobramycin, ofloxacin, ciprofloxacin).

The term “antibody” refers to an immunoglobulin protein, or functional portion thereof, including a polyclonal antibody, a monoclonal antibody, a chimeric antibody, a hybrid antibody, a single chain antibody, a mutagenized antibody, a humanized antibody, and antibody fragments that comprise an antigen binding site (e.g., Fab and Fv antibody fragments).

The term “small molecule” as used herein refers to a compound, for example an organic compound, with a molecular weight of less than about 1,000 Daltons, such as less than about 750 Daltons, or in some embodiments less than about 600 Daltons, and in other embodiments less than about 500 Daltons. A small molecule can have a computed log octanol-water partition coefficient in the range of about −4 to about +14, such as in the range of about −2 to about +7.5.

The term “biologically active agent” refers to a composition that causes an observable change in the structure, function, or composition of a cell upon uptake by the cell. Observable changes include increased or decreased expression of one or more mRNAs, increased or decreased expression of one or more proteins, phosphorylation of a protein or other cell component, inhibition or activation of an enzyme, inhibition or activation of binding between members of a binding pair, an increased or decreased rate of synthesis of a metabolite, increased or decreased cell proliferation, and the like.

The term “drug” as used herein refers to any substance having biological or detectable activity. Thus, the term “drug” includes a pharmaceutical agent, a diagnostic agent, a binding agent, a heterologous agent, and combinations thereof.

The term “therapeutic agent” refers to any composition that can be used to treat or prevent a condition in a subject in need thereof, or to the benefit of the intended subject.

The term “detectable label” refers to a composition that can be detected following membrane translocation of the label.

The term “binding agent” refers to a composition that specifically binds a target molecule. Representative binding agents include antibodies, targeting peptides, ligands, cell adhesion ligands, etc.

The term “binding” refers to an affinity between two molecules, for example, a ligand and a receptor. As used herein, “binding” means a preferential binding of one molecule for another in a mixture of molecules. The binding of a ligand to a receptor can be considered specific if the binding affinity is about 1×10⁴ M⁻¹ to about 1×10⁶ M⁻¹ or greater.

The phrase “specifically (or selectively) binds,” as used herein to describe the binding capacity of a peptide, refers to a binding reaction which is determinative of the presence of the protein in a heterogeneous population of proteins and other biological materials.

The term “heterologous agent” refers to any molecule that originates from a source foreign to an intended host cell or, if from the same source, is modified from its original form. A heterologous agent can be or comprise a molecule that is endogenous to the particular host cell but has been modified, for example by mutagenesis. A heterologous agent also includes non-naturally occurring levels of a native molecule.

The term “in vivo, ” as used herein to describe imaging or detection methods, refer to generally non-invasive methods such as scintigraphic methods, magnetic resonance imaging, ultrasound, or fluorescence, each described briefly herein below. The term “non-invasive methods” does not exclude methods employing administration of a contrast agent to facilitate in vivo imaging. For in vivo detection, useful detectable labels include a fluorophore, an epitope, or a radioactive label, also described briefly herein below.

The term “in vitro” refers to cells that are maintained in culture, including primary culture or culture of a cell line. Thus, cell removed from the body and maintained in culture, for example for the purpose of manipulation using ex vivo therapy techniques, is a cell in vitro.

The term “in vivo” refers to cell in a body.

The term “about”, as used herein when referring to a measurable value such as an amount, a signal intensity, a transport rate, etc., is meant to encompass variations of ±20% or, in some embodiments, ±10%, such as ±5%, or such as ±1%, or such as ±0.1% from the specified value, as such variations are appropriate to perform the disclosed methods.

The terms “a,” “an,” and “the” are used in accordance with convention in the art to refer to one or more.

II. Membrane-Translocating Peptides

TAT has been shown to translocate through the cell membranes via a receptor-independent pathway (Casellas et al., 1988; Nagahara et al., 1998). Indeed, peptides with more than 6 arginine sequences have been reported to follow the same pathway of TAT, suggesting that the conventional endocytosis pathway does not play a crucial role in the cell translocation for these arginine-rich peptides (Futaki et al., 2001; Morris et al., 2001; Wender et al., 2000).

The present invention provides translocating peptides derived from protamine. Non-toxic low molecular weight protamine fragments are described in Byun et al. (1999) Thromb Res 94:53-61; Chang et al. (2001a) AAPS PharmSci 3: article 18; Chang et al. (2001b) AAPS PharmSci 3: article 17; and Lee et al. (2001) AAPS PharmSci 3: article 19. Sequence analysis of the peptides showed that two of the LMWP peptides (TDSP4 & TDSP5) have significant amino acid similarity to the membrane-translocating peptide TAT5 (Chang et al., 2001a). Representative LMWP peptides are set forth as SEQ ID NOs: 1-4. The LMWP membrane-translocating peptides share a similar structural scaffold of arginine clusters in the middle and a non-arginine residue at the N-terminal of the peptide sequence.

As disclosed herein, these LMWP peptides also behave similarly to TAT5 in their ability to transverse biological membranes efficiently via a receptor-independent endocytotic mechanism. The LMWP peptides can translocate a variety of agents, including small molecules and nucleic acids. See Examples 3, 6, and 9. Thus, methods for cell translocation using LMWP peptides have broad utility both in vitro and in vivo, as described further herein below.

The LMWP peptides differ from existing membrane-translocating peptides such as TAT5, including (1) known clinical performance and safety, and (2) methods for rapid and economical production of large quantities. LMWP peptides have previously been used as a substitute for protamine in clinical heparin neutralization. See Byun et al. (1999) Thromb Res 94: 53-61; Chang et al. (2001) AAPS PharmSci 3: article 18; Chang et al. (2001) AAPS PharmSci 3: article 17; Lee et al. (2001) AAPS PharmSci 3: article 19. The known clinical performance and safety profile of the LMWP peptides is a significant advantage to their use as membrane-translocating peptides, as disclosed herein. The LMWP peptides possess significantly less antigenicity (i.e., the ability of a substance to be recognized by an antibody), mutagenicity (i.e., the ability of a substance to induce the production of antibodies), complement-activating activity, and other cationic polymer-associated hemodynamic or hematologic toxic side effects when compared to the parent protamine, which is already FDA-approved and widely used in clinical settings. See Liang et al. (2003) Biochemistyr (Moscow) 68(1):116-20; Lee et al. (2001) AAPS PharmSci 3: article 19; Tsui et al. (2001) Thromb Res 101: 417-20. The LMWP fragments can be derived directly from native protamine by enzymatic digestion with thermolysin, and thus are readily produced in mass quantities within short time duration and low costs. While the LMWP peptides can be prepared by any method known in the art, the peptides do not require synthetic techniques.

II.A. Preparation of LMWP Peptides

As noted herein above, the LMWP peptides disclosed herein can be produced en masse via proteolytic digestion of a protamine molecule. The LMWP peptides can be derived directly from protamine using a “separation-free” enzymatic degradation (i.e., by utilizing immobilized thermolysin) as well as one single step of isolation and purification using a heparin affinity chromatography, and thus can be readily produced in mass quantity within a short duration of time (e.g, 1 g/week based on our own laboratory scale). Representative methods are described in Example 1.

Thermolysin is a metalloendopeptidase that hydrolyzes peptide bonds on the imino side of large hydrophobic residues such as isoleucine and phenylalanine. Thermolysis is typically derived from Bacillus thermoproteolyticus, and variants are also found in Micrococcus caseolyticus and Aspergillus oryzae.

Peptides of the present invention, including peptoids, can also be synthesized by any of the techniques that are known to those skilled in the art of peptide synthesis. A summary of representative techniques can be found in Stewart & Young (1984) Solid Phase Peptide Synthesis, Pierce Chemical Co., Rockville, Ill.; Merrifield (1969) Adv Enzymol Relat Areas Mol Biol 32:221-296; Fields & Noble (1990) Int J Pept Protein Res 35:161-214; Bodanszky (1993) Principles of Peptide Synthesis, Springer-Verlag, New York; Andersson et al. (2000) Biopolymers 55:227-50; and in U.S. Pat. Nos. 6,015,561, 6,015,881, 6,031,071, and 4,244,946. In addition, peptides comprising a specified amino acid sequence can be purchased from commercial sources (e.g., Biopeptide Co., LLC of San Diego, Calif. and PeptidoGenics of Livermore, Calif.).

A peptide mimetic is identified by assigning a hashed bitmap structural fingerprint to the peptide based on its chemical structure, and determining the similarity of that fingerprint to that of each compound in a broad chemical database. The fingerprints can be determined using fingerprinting software commercially distributed for that purpose by Daylight Chemical Information Systems, Inc. (Mission Viejo, Calif.) according to the vendor's instructions. Representative databases include but are not limited to SPREI'95 (InfoChem GmbH of München, Germany), Index Chemicus (ISI of Philadelphia, Pa.), World Drug Index (Derwent of London, United Kingdom), TSCA93 (United States Environmental Protection Agency), MedChem (Biobyte of Claremont, Calif.), Maybridge Organic Chemical Catalog (Maybridge of Cornwall, England), Available Chemicals Directory (MDL Information Systems of San Leandro, Calif.), NCI96 (United States National Cancer Institute), Asinex Catalog of Organic Compounds (Asinex Ltd. of Moscow, Russia), and NP (InterBioScreen Ltd. of Moscow, Russia). A peptide mimetic of an LMWP peptide is selected as comprising a fingerprint with a similarity (Tanamoto coefficient) of at least 0.85 relative to the fingerprint of the LMWP peptide, which is capable of traversing cell membranes.

A peptide mimetic can also be designed by: (a) identifying the pharmacophoric groups responsible for the targeting activity of a peptide; (b) determining the spatial arrangements of the pharmacophoric groups in the active conformation of the peptide; and (c) selecting a pharmaceutically acceptable template upon which to mount the pharmacophoric groups in a manner that allows them to retain their spatial arrangement in the active conformation of the peptide. For identification of pharmacophoric groups responsible for targeting activity, mutant variants of the peptide can be prepared and assayed for targeting activity. Alternatively or in addition, the three-dimensional structure of a complex of the peptide and its target molecule can be examined for evidence of interactions, for example the fit of a peptide side chain into a cleft of the target molecule, potential sites for hydrogen bonding, etc. The spatial arrangements of the pharmacophoric groups can be determined by NMR spectroscopy or X-ray diffraction studies. An initial three-dimensional model can be refined by energy minimization and molecular dynamics simulation. A template for modeling can be selected by reference to a template database and will typically allow the mounting of 2-8 pharmacophores. A peptide mimetic is identified wherein addition of the pharmacophoric groups to the template maintains their spatial arrangement as in the peptide. Techniques for the design and preparation of peptide mimetics can be found in U.S. Pat. Nos. 5,811,392; 5,811,512; 5,578,629; 5,817,879; 5,817,757; and 5,811,515.

Any peptide or peptide mimetic of the present invention can be used in the form of a pharmaceutically acceptable salt. Suitable acids which are capable of the peptides with the peptides of the present invention include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. HCl and TFA salts are readily available and convenient to use.

Suitable bases capable of forming salts with the LMWP peptides of the present invention include inorganic bases such as sodium hydroxide, ammonium hydroxide, potassium hydroxide and the like; and organic bases such as mono-di- and tri-alkyl and aryl amines (e.g. triethylamine, diisopropyl amine, methyl amine, dimethyl amine and the like), and optionally substituted ethanolamines (e.g. ethanolamine, diethanolamine and the like).

II.B. Variations

The term “peptide” encompasses any of a variety of forms of peptide derivatives, including amides, conjugates with proteins, cyclized peptides, polymerized peptides, conservatively substituted variants, analogs, fragments, peptoids, chemically modified peptides, and peptide mimetics. Thus, an LMWP peptide of the present invention can be subject to various changes, substitutions, insertions, and deletions, wherein such changes provide for certain advantages in its use as a membrane-translocating peptide. Representative methods for assessing membrane-translocating activity are described in Examples 3, 6, and 9.

Peptides of the invention can comprise naturally occurring amino acids, synthetic amino acids, genetically encoded amino acids, non-genetically encoded amino acids, and combinations thereof. Peptides can include both L-form and D-form amino acids.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine.

The term “conservatively substituted variant” refers to a peptide, e.g., an LMWP-like peptide comprising an amino acid in which one or more residues have been conservatively substituted with a functionally similar residue and which displays the membrane-translocating activity as described herein. The phrase “conservatively substituted variant” also includes peptides wherein a residue is replaced with a chemically derivatized residue.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

LMWP peptides used in the methods of the present invention also include peptides comprising one or more deletions of residues relative to the sequence of an LMWP peptide, so long as the requisite membrane-translocating activity of the peptide is maintained. The term “fragment” refers to a peptide comprising an amino acid residue sequence shorter than that of a peptide disclosed herein.

LMWP peptides can also include one or more additions of residues relative to the sequence of an LMWP peptide, so long as the requisite membrane-translocating activity of the peptide is maintained. For example, membrane-translocating peptides or about 6 to about 50 residues or more, can be prepared, wherein the peptide includes the amino acid sequence of any one of SEQ ID NOs: 1-4.

Additional residues can also be added at either terminus of a peptide for the purpose of providing a “linker” by which the peptides of the present invention can be conveniently affixed to a label or solid matrix, or carrier. Amino acid residue linkers are usually at least one residue and can be 40 or more residues, more often 1 to 10 residues, but do alone not constitute peptide analogs having receptor binding activity. Typical amino acid residues used for linking include tyrosine, cysteine, lysine, glutamic and aspartic acid, or the like. In addition, a peptide can be modified by terminal-NH₂ acylation (e.g., acetylation, or thioglycolic acid amidation) or by terminal-carboxylamidation (e.g., with ammonia, methylamine, and the like terminal modifications), or cyclized. Terminal modifications are useful, as is well known, to reduce susceptibility by proteinase digestion, and therefore serve to prolong half life of the peptides in solutions, particularly biological fluids where proteases can be present.

The term “peptoid” as used herein refers to a peptide wherein one or more of the peptide bonds are replaced by pseudopeptide bonds including but not limited to a carba bond (CH₂—CH₂), a depsi bond (CO—O), a hydroxyethylene bond (CHOH—CH₂), a ketomethylene bond (CO—CH₂), a methylene-oxy bond (CH₂—O), a reduced bond (CH₂—NH), a thiomethylene bond (CH₂—S), a thiopeptide bond (CS—NH), and an N-modified bond (—NRCO—). See e.g., Corringer et al. (1993) J Med Chem 36:166-72, Garbay-Jaureguiberry et al. (1992) Int J Pept Protein Res 39:523-7, Tung et al. (1992) Pept Res 5:115-8, Urge et al. (1992) Carbohydr Res 235:83-93, and Pavone et al. (1993) Int J Pept Protein Res 41:15-20.

The term “peptide mimetic” as used herein refers to a ligand that mimics the biological activity of an LMWP peptide, by substantially duplicating the membrane-translocating activity of the LMWP peptide, but it is not a peptide or peptoid. Preferably, a peptide mimetic has a molecular weight of less than about 700 Daltons.

III. Preparation of LMWP Conjugates, LMWP Complexes, and LWMP Fusion Proteins

The present invention further provides LMWP compositions capable of cellular translocation, and methods for preparing the same. The LMWP compositions include LMWP conjugates, LMWP complexes, and LMWP fusion proteins. In a representative embodiment of the invention, an LMWP composition comprises an LMWP peptide and a cargo molecule. An LMWP composition can comprise one or more LMWP peptides, including a combination of two or more different LMWP peptides.

III.A. LMWP Conjugates

The term “peptide conjugate,” as used herein, refers to a composition prepared by chemical reaction of an LMWP peptide with a cargo molecule, for example, via a carbamate linkage, an ester linkage, a thioether linkage, a disulfide linkage, or a hydrazone linkage. Optionally, a chelator can be used to facilitate linkage of an LMWP peptide and a drug or other cargo molecule.

Various functional groups (hydroxyl, amino, halogen, etc.) can be used to attach the cargo molecule to the LMWP peptide. The functional groups can be present at a non-active site of the cargo molecule, such as when the cargo is to remain attached to the LMWP peptide after delivery.

Coupling reactions can be performed by known coupling methods in any of an array of solvents, such as N,N-dimethyl formamide (DMF), N-methyl pyrrolidinone, dichloromethane, water, etc. Exemplary coupling reagents include O-benzotriazolyloxy tetramethyluronium hexafluorophosphate (HATU), dicyclohexyl carbodiimide, bromo-tris(pyrrolidino) phosphonium bromide (PyBroP), etc. Other reagents can also be included, such as, for example, N,N-dimethylamino pyridine (DMAP), 4-pyrrolidino pyridine, N-hydroxy succinimide, N-hydroxy benzotriazole, etc.

Chemical linkage of a drug to an LMWP peptide can comprise a stable linkage, for example a covalent bond. Alternatively, as desired for a particular application, the linkage can be labile, such as a disulfide bond, an acid-labile linkage, or an enzyme-labile linkage. For example, a drug attached to an LMWP peptide via a disulfide bond is redox active, such that it is stable in the serum and is released upon entry into the reducing environment of the cell cytosol. Similarly, a drug or chelator can be attached to an LMWP peptide via a functional group that effects drug/chelator release in the lysosome.

Representative methods for preparing an LMWP conjugate are described in Example 5, which describes preparation of LMWP-gelonin. Gelonin is a plant ribosome-inactivating protein (RIP) with n-glycosidase activity similar to that of ricin A chain. The LMWP-gelonin conjugate was efficiently translocated across cell membranes and showed kinetics of uptake into cells that was similar to TAT-gelonin (Example 6). While LMWP or gelonin alone showed minimal or no cytotoxicity, uptake of the LMWP-gelonin conjugate induced cell toxicity and antitumor activity in vivo (Example 7).

Using similar methods, polymeric LMWP conjugates can be prepared to included small molecule drugs (e.g., antitumor drugs such as doxorubicin or antibiotics such as ofloxacin).

III.B. LMWP Complexes

The term “LMWP complex,” as used herein, refers to a composition prepared by association of an LMWP peptide and a drug via an ionic interaction. As disclosed herein, peptide complexes are readily formed by association of an LMWP peptide and a nucleic acid.

The term “complexing,” as used herein, refers to a process whereby a nucleic acid is directly bound to an LMWP peptide via an ionic interaction. The process of “complexing” can also include condensation of the nucleic acid, as disclosed herein. The nucleic acid of an LMWP complex is described as “complexed with” an LMWP peptide.

Example 8 describes representative methods for preparing an LMWP peptide complex with DNA. Gel-retardation results demonstrated formation of a stable complex between TDSP5 and DNA at a charge ratio (−/+)=1:1. Simple complexation of pDNA with TDSP5 yielded nano-sized particles even at a low charge ratio. Thus, LMWP peptides are capable of condensing DNA to a size appropriate for cell delivery. See e.g., Midoux & Monsigny (1999) Bioconjug Chem 10:406-11 and Zauner et al. (1996) Biotechniques 20:905-13. Previous studies have demonstrated that feasibility of utilizing protamine as a possible gene carrier due to its ability to condense pDNA (Wadhwa et al., 1997). As disclosed herein, for the first time, the nucleic acid condensing activity of LMWP is similar to that of protamine. In addition, LMWP increases the surface potential of the pDNA complexes, thereby enhancing its interaction with the cell surface membrane.

pDNA/LMWP complexes so-prepared were able to traverse cell membranes. Confocal microscopy analyses show that FITC-labeled DNA molecules, which are complexed to LMWP, accumulate in the nucleus and cytoplasm. The kinetics of cell transfection is comparable or improved when compared to polyfection with PEI and with DNA/Lipofectamine complex, which result in nuclear distribution of DNA after 4 hours of incubation. See e.g., Benimetskaya et al. (2002) Bioconjug Chem 13:177-87 and Wightman et al. (2001) J Gene Med 3:362-72. While the inventors do not wish to be bound to any particular mode of operation, early nuclear localization of pDNA/LMWP complexes can be explained by direct, receptor-independent uptake, whereas other cationic polyplexes are delivered by the receptor-mediated (or adsorptive) endocytosis that requires a further endosomal release step inside the cell. Nucleic acids can remain complexed with LMWP peptides once inside the cell, or the nucleic acids can dissociate from the LMWP.

The pDNA/LMWP complexes showed a significantly higher transfection efficiency comparing to that of the pDNA/PEI complex. As known in the art, cationic pDNA/PEI complexes are taken into the cells via an endocytotic mechanism, the complexes are disrupted in endosomes, and pDNA is then released into the cytosol. In contrast, LMWP-condensed pDNA complexes are taken directly into the cytosol and are protected from lysosomal and DNase degradation (Example 8), thereby achieving a significantly increased transfection efficiency (Example 9).

III.C. LMWP Fusion Proteins

The term “fusion protein,” as used herein to describe an LMWP composition, refers to a recombinantly produced peptide comprising: (1) an LMWP peptide, and (2) a peptide or polypeptide of interest (i.e., a cargo peptide or polypeptide). Optionally, a fusion protein can also include a linker between the LMWP peptide and the peptide/polypeptide of interest.

Where the cargo molecule comprises a peptide or polypeptide, the present invention further provides methods for recombinant production of a fusion protein comprising an LMWP peptide. Thus, the invention further relates to methods for using a nucleic acid sequence encoding an LMWP peptide to genetically engineer membrane-permeable polypeptides, including peptides and proteins. Briefly, an expression vector is designed so that the DNA sequence encoding an LMWP peptide will be positioned N-terminal or C-terminal to a target sequence in the a reading frame suitable for expression of LMWP and the target sequence as a fusion protein. The target sequence encodes a polypeptide, which is desired to be made membrane-permeable. The LMWP sequence of the fusion protein mediates cellular import of the fusion protein.

Expression system vectors are known to those of skill in the art. The expression vector chosen by one of skill in the art can include regulatory elements appropriate for expression, including promoter and enhancer elements, termination signals, and sequences required for translation. Vectors can also include restriction sites that simplify cloning and/or sequences that assist in purification. Suitable vectors include but are not limited to viruses such as vaccinia virus or adenovirus, baculovirus vectors, yeast vectors, bacteriophage vectors (e.g., lambda phage), plasmid and cosmid DNA vectors, transposon-mediated transformation vectors, and derivatives thereof.

An expression vector host cell system can be chosen from among a number of such systems that are known to those of skill in the art. Fusion proteins can be expressed in bacteria, yeast, eukaryotic cells (e.g., mammalian cells, amphibian cell, and insect cells), or cell-free expression systems. A host cell strain can be chosen which modulates the expression of the recombinant sequence, or modifies and processes the gene product in the specific fashion desired. For example, different host cells have characteristic and specific mechanisms for the translational and post-transactional processing and modification (e.g., glycosylation, phosphorylation of proteins). Appropriate cell lines or host systems can be chosen to ensure the desired modification and processing of the foreign protein expressed. For example, expression in a bacterial system can be used to produce a non-glycosylated core protein product, and expression in yeast can produce a glycosylated product.

Methods for recombinant production of fusion proteins are known in the art. Standard recombinant DNA and molecular cloning techniques can be found in, for example, Sambrook et al. (eds.) (1989) Molecular Cloning: A Laboratory Manual. Cold Spring Harbor Laboratory Press, Cold Spring Harbor, N.Y.; Silhavy et al. (1984) Experiments with Gene Fusions. Cold Spring Harbor Laboratory Press, Cold Spring Harbor, N.Y.; Glover & Hames (1995) DNA Cloning: A Practical Approach 2nd ed. IRL Press at Oxford University Press, Oxford/N.Y.; and Ausubel (ed.) (1995) Short Protocols in Molecular Biology, 3rd ed. Wiley, N.Y., among other places.

IV. Cellular Non-Toxicity of LMWP Compositions

As noted previously, LMWP peptides are nontoxic in vitro as well as in vivo. Previous studies have demonstrated that LMWP peptides retain the heparin-neutralizing capability and yet are devoid of the toxic effects of protamine. In addition, LMWP peptides display a significantly reduced level of immunogenicity (i.e., the ability to induce the production of antibodies) and antigenicity (i.e., the ability to cross-react with anti-protamine antibodies), which are responsible for protamine-induced immunotoxicity. See Chang et al. (2001) AAPS PharmSci 3: article 18; Chang et al. (2001) AAPS PharmSci 3: article 17; Lee et al. (2001) AAPS PharmSci 3: article 19.

Thus, the present invention provides that non-toxic LMWP conjugates and LMWP complexes can be prepared. See Examples 4, 7, and 10. The term “non-toxic” generally refers to a quality of not inducing cellular harm or lethality.

In the fields of gene transfection and gene therapy, the cellular toxicity of cationic polymers such as PEI is a significant concern in their uses as gene carriers. Although the toxicity of the complex may be relieved due to charge neutralization of the cationic polymer when complexed with DNA, the polymer would remain toxic after the detachment of pDNA. In this regard, the lack of toxicity of LMWP peptides even without DNA complexation is a substantial advantage to the use of LMWP for delivery of nucleic acids.

V. Uses of Membrane-Translocating Peptides

The present invention discloses new methods that employ membrane-translocating peptides. The utility of the LMWP peptides lies in their ability to translocate across membranes, whereby cargo molecules attached to, fused with, or otherwise associated with the LMWP peptides are also translocated across cell membranes. Thus, the methods have broad utility in methods for cellular delivery. The cells can be in vitro or in vivo.

The LMWP compositions of the invention are useful for transporting biologically active agents across cell or organelle membranes, when the agents are of the type that require trans-membrane transport to exhibit their biological effects; and that do not exhibit their biological effects primarily by binding to a surface receptor, i.e., such that entry of the agent does not occur. Further, the LMWP compositions useful for transporting biologically active agents of the type that require trans-membrane transport to exhibit their biological effects, and that by themselves (without conjugation to a transport polymer or some other modification), are unable, or only poorly able, to enter cells to manifest biological activity.

The LMWP compositions of the present invention can be used as a vehicle for in vitro cell transformation. The term “transformation,” as used herein, refers to delivery of a heterologous agent to a cell. The term “transformation system” refers to a host cell comprising a heterologous agent.

The term “heterologous agent” refers to any molecule that originates from a source foreign to an intended host cell or, if from the same source, is modified from its original form. A heterologous agent can comprise a molecule that is endogenous to the particular host cell but has been modified, for example by mutagenesis. A heterologous agent also includes non-naturally occurring levels of a native molecule.

In one embodiment of the invention, a transformation system is useful for production of heterologous nucleic acids and proteins. For example, LMWP complexes as described herein can be used to establish an expression system. The term “expression system” refers to a host cell comprising a heterologous nucleic acid and the polypeptide encoded by the heterologous nucleic acid. LWMP peptides, conjugates, complexes, and fusion proteins can be administered to cells or membranes in vitro by addition to the culture medium.

In another embodiment of the invention, a transformation system can be used as an in vitro assay or screening method. For example, LMWP conjugates can be prepared from one or more test substances. The conjugates are contacted with a cell that exhibits an observable change or detectable signal upon uptake of the conjugate into the cell, target tissue or pathogenic biofilm layer, such that the magnitude of the change or signal is indicative of the efficacy of the conjugate with respect to an associated biological activity. This method can be used to test the activities of test substances that by themselves are unable, or poorly able, to enter cells to manifest biological activity.

LMWP compositions comprising a therapeutic agent are useful in drug delivery methods. Representative therapeutic agents include but are not limited to a therapeutic gene, a vaccine, an immunomodulatory agent, an anti-cancer agent, an anti-angiogenic agent, a chemotherapeutic agent, an antibiotic agent (e.g., ofloxacin, tobramycin), a cytotoxin (e.g., gelonin), a radionuclide, etc.

Representative methods for preparation of an LMWP comprising an anti-cancer agent, LMWP-gelonin, are described in Example 5. Administration and cytotoxic activity of LMWP-gelonin are described in Example 7.

Other target cells are prokarytoic cells, for example bacterial cells, including bacterial cells of a biofilm. Bacterial biofilms are frequently observed on the surfaces of tissue and biomaterials at the site of persistent infections. Biofilm formation is a major cause of implant failure and can limit the duration of an implanted medical device. Treatment of an infection after biofilm formation is difficult, in part because the biofilm is a dense structure that inhibits penetration of antibiotics. See e.g., Walters et al. (2003) Antimicrob Agents Chemother 47(1):317-323; Anderl et al. (2000) Antimicrob Agents Chemother 44(7):1818-1824; Darveau et.al. (1997) Periodontol 2000 14:12-32. The present invention provides LMWP-conjugates comprising one or more antibiotics, which can be used to penetrate a biofilm layer.

LWMP compositions comprising a detectable label are useful for detection and/or imaging methods. For example, detection or imaging of a target molecule in a cell can be accomplished using an LMWP composition comprising a detectable label and a binding agent that specifically binds to the target molecule. Whole body or whole tissue imaging can be performed using LMWP compositions that lack a binding agent. Detection methods that employ LMWP compositions are suited for detection or imaging of live cells or subjects because fixation and permeabilizing reagents are not required. Representative detectable labels include but are not limited to a radionuclide (for scintigraphic imaging), contrast agents such as paramagnetic or superparamagnetic metal ions and iron oxide particles (for magnetic resonance imaging), gas microbubbles (for ultrasonic imaging), fluorescent labels, epitope labels, and radioactive labels. Methods for preparing labeled peptides are known in the art. Imaging methods for visualization of labeled peptides are also known in the art. Representative methods for fluorescent labeling and detection of an LMWP composition are described in Examples 3, 6, and 9.

The compositions of the invention can be formulated according to known methods to prepare pharmaceutical compositions. Suitable formulations for administration to a subject include aqueous and non-aqueous sterile injection solutions which can contain one or more adjuvants, anti-oxidants, buffers, bacteriostats, antibacterial and antifungal agents (e.g., parabens, chlorobutanol, phenol, ascorbic acid, an thimerosal), solutes that render the formulation isotonic with the bodily fluids of the intended recipient (e.g., sugars, salts, and polyalcohols), suspending agents and thickening agents. Suitable solvents include water, ethanol, polyol (e.g., glycerol, propylene glycol, and liquid polyethylene glycol), and mixtures thereof. The formulations can be presented in unit-dose or multi-dose containers, for example sealed ampoules and vials, and can be stored in a frozen or freeze-dried (lyophilized) condition requiring only the addition of sterile liquid carrier immediately prior to use.

Formulations according to the invention are buffered to a pH of from about 5 to about 7, such as about 6. Suitable buffers include those which are physiologically acceptable upon administration by inhalation. Such buffers include, for example, citric acid buffers and phosphate buffers, of which phosphate buffers are preferred. Useful phoshate buffers include monosodium phosphate dihydrate and dibasic sodium phosphate anhydrous.

An LMWP composition of the invention can be formulated to confer qualities appropriate for its intended use. For example, LMWP compositions can further comprise protein stabilizers. See e.g., U.S. Pat. No. 5,711,968, (use of zinc to stabilize recombinant human growth hormone and recombinant α-interferon in microspheres) and U.S. Pat. No. 5,674,534 (use of ammonium sulfate to stabilize erythropoietin during release from hydrated microspheres). An LMWP composition can also comprise nano-carriers such as magnetic nano-particles (Josephson et al., 1999), liposomes (Torchilin et al. (2001) Proc Natl Acad Sci USA 98: 8786-91), nanospheres (U.S. Pat. Nos. 6,207,195 and 6,177,088), and nanosuspensions (U.S. Pat. No. 5,858,410).

The LMWP compositions of the present invention can be delivered to eukaryotic or prokaryotic cells, including cultured cells and cells of an organism. Representative eukaryotic cells include mammalian cells such as human cells. Prokaryotic cells include bacterial cells, either in culture or in an organism having a bacterial infection.

For in vitro applications, LWMP peptides, conjugates, complexes, and fusion proteins can be administered to cells or membranes in vitro by addition to the culture medium. For in vivo applications, LWMP peptides, conjugates, complexes, and fusion proteins can be delivered by standard methods utilized for protein/drug delivery, including parenteral administration, intravenous administration, intratumoral administration, topical administration, aerosol administration or inhalation, oral administration. Encapsulated forms are often used for oral administration, and suppositories are often used in rectal and vaginal administration.

For in vitro or in vivo use, LWMP peptides, conjugates, complexes, and fusion proteins are provided in an effective amount. The term “effective amount” is used herein to describe an amount of a LMWP composition of the invention that is sufficient to elicit a desired biological response. For diagnostic applications, a detectable amount of a composition of the invention is administered to a subject. A “detectable amount,” as used herein to refer to a diagnostic composition, refers to a dose of an LMWP composition that can be determined following administration to a cell culture or to a subject. Uptake of the fusion protein is dependent upon the external concentration of the fusion protein and the period of application, therefore the internal concentration of protein can be controlled by controlling administration to the extracellular environment.

Actual dosage levels of active ingredients in a composition of the invention can be varied so as to administer an amount of the composition that is effective to achieve the desired diagnostic or therapeutic outcome for a particular subject. Administration regimens can also be varied. When administered by injection, a single injection or multiple injections can be used. The selected dosage level and regimen will depend upon a variety of factors including the activity of the therapeutic composition, formulation, the route of administration, combination with other drugs or treatments, the disease or disorder to be detected and/or treated, and the physical condition and prior medical history of the subject being treated. Determination and adjustment of an effective amount or dose, as well as evaluation of when and how to make such adjustments, are known to those of ordinary skill in the art of medicine. In one embodiment, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

### EXAMPLES

The following Examples are included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Preparation of LMWP Peptides by Proteolytic Digestion

LMWP fragments were derived from natural protamine by thermolysine digestion essentially as described by Chang et al. (2001b) AAPS PharmSci 3: article 17. In brief, thermolysin and protamine were mixed and incubated for 30 minutes at room temperature, followed by quenching thermolysin activity with 50 mM EDTA. Thermolysin was then removed by ultrafiltration using a YM3 membrane (MWCO 3000), and the filtrate was subject to lyophilization. Since the lyophilized LMWP preparation contained a mixture of small peptide impurities, it was further purified using a heparin affinity chromatography.

Five different fractions were isolated and purified. The fractions were denoted TDSP (thermolysin-digested segmented protamine) 1 to 5, depending on their elution order from a heparin affinity column. The molecular weight and amino acid sequence of each isolated LMWP peptide were determined by MALDI-TOF MS analysis, which was performed by the Protein and Carbohydrate Research Center at the University of Michigan. The amino acid sequences of TDSP2, TDSP3, TDSP4, and TDSP5 are set forth as SEQ ID NOs: 1, 2, 3, and 4, respectively.

### Example 2

### Fluorescent Labeling of Peptides

Peptides were labeled with fluorescein isothiocyanate (FITC) at their N-terminals. In brief, the peptide solution (pH 9.3, carbonate buffer) was reacted, in 1:2 molar ratio, with a FITC solution (in dimethylformamide) overnight in the dark at room temperature. The labeling reaction was monitored by HPLC of the absorbance change at 215 nm of the peptide peak. Labeled peptides were purified by HPLC (purity>95%), lyophylized in the dark, and then stored at −20° C. in the dark until further use.

### Example 3

### Cell Translocation Activity of LMWP Peptides

The cell internalization activity of each of TDSP2 (SEQ ID NO: 1), TDSP3 (SEQ ID NO:2), TDSP4 (SEQ ID NO:3), and TDSP5 (SEQ ID NO:4) were examined. The membrane-translocating activity of TDSP1 was not examined on the basis that it possessed less than 6 amino acid residues, which is thought to be a minimal number of residues that can support cell transduction. See Futaki et al. (2001) J Biol Chem 276: 5836-40.

Cell lines including 293T, HeLa, CT26, human MCF-7 cell lines were obtained from American Type Culture Collection (ATCC, of Rockville, Md.). They were cultured in either DMEM medium (293T, HeLa) or RPMI1640 (CT26, MCF-7) containing 10% FBS, 100 units/mL penicillin-streptomycin mixture, 2.2 mg/mL sodium bicarbonate, at 37° C. in a humidified atmosphere containing 5% CO₂.

FITC-labeled LMWP peptides or control peptides were added to 10⁴ cells cultured in LAB-TEK™ chambered cover glasses (Lab-Tek Plastics company of Westmont, Ill.). After 30 minutes incubation with FITC-labeled LMWP peptides cells were washed with PBS and fixed with 1% paraformaldehyde for 20 minutes. The fixed cells were washed again with PBS, mounted in a PBS/glycerin mixture (1:1, v/v) containing 2.5% DABCO (1,4-diazobicyclo-(2,2,2)octane) as an antifading agent , and kept at 4° C. for at least one hour before evaluation. Confocal laser scanning microscopy was performed on an inverted LSM 510 model laser scanning microscope (Carl Zeiss of Gottingen, Germany).

To quantify membrane translocation of peptides, the cellular uptake of each peptide was measured by the mean fluorescent signal for 10,000 cells collected. Cells were seeded at a density of 1×10⁶ cells per well in 6 well plates containing 1.5 ml culture medium for 24 hours and then incubated with the FITC-labeled LMWP peptides for 30 minutes. To study cell internalization in the presence of serum, the LMWP peptides was dissolved in DMEM in the absence of serum followed by the addition of 10% FBS. After incubation, the cells were washed and treated with trypsin. The cells were then fixed with 1% paraformaldehyde and washed with PBS. FACS analysis was conducted using a flow cytometer (Becton Dickinson of San Jose, Calif.) equipped with a 488 nm air-cooled argon laser. The filter settings for emission were 530/30 nm bandpass (FL1) for FITC. The fluorescence of 10,000 vital cells was acquired and data was visualized in logarithmic mode.

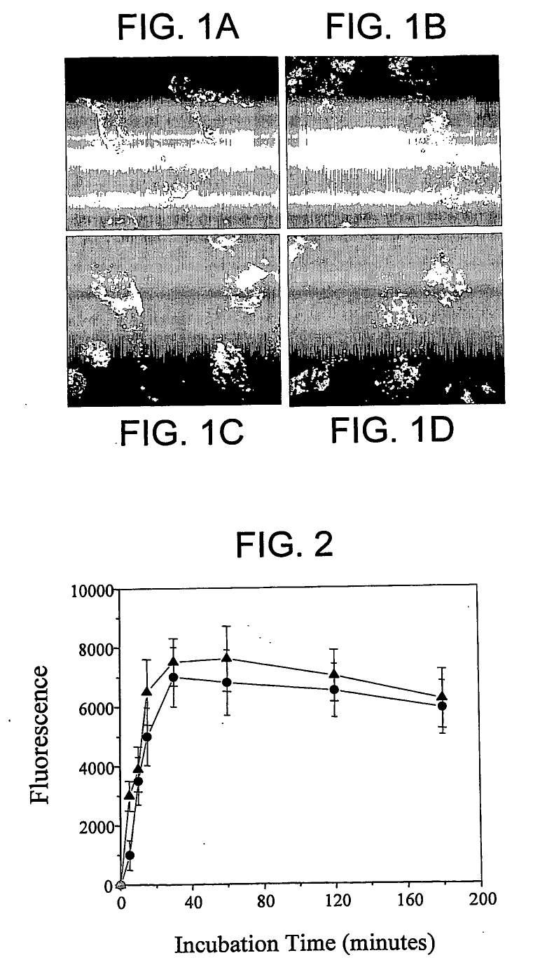

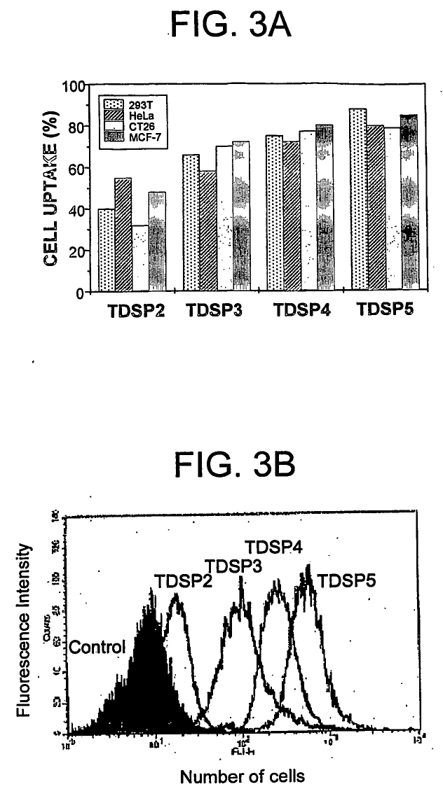

Internalization of the LMWP was monitored by confocal microscopy after 15 minutes and 60 minutes incubation of the peptides with the cells. LMWP internalized into the cell as efficiently as the TAT peptides. In less than 15 minutes, LMWP and TAT peptides were detected mainly in cytosol and some perinuclear localization (FIGS. 1A, 1C). After 1 hour, both peptides were seen to translocate through cell membranes and accumulate in the cytoplasm and nucleus (FIGS. 1B, 1D). Almost all of the cell population exhibited a high fluorescent intensity, demonstrating efficient cell internalization by both LMWP and TAT. Similar results were observed for TDSP4, which has a substantially similar amino acid sequence to that of TDSP5. FACS analysis of FITC-labeled LMWP peptides showed rapid uptake and sustained detection of the labeled peptides in cells (FIG. 2). The timecourse of cellular uptake of LMWP peptides was similar to that displayed by TAT peptides (FIG. 2).

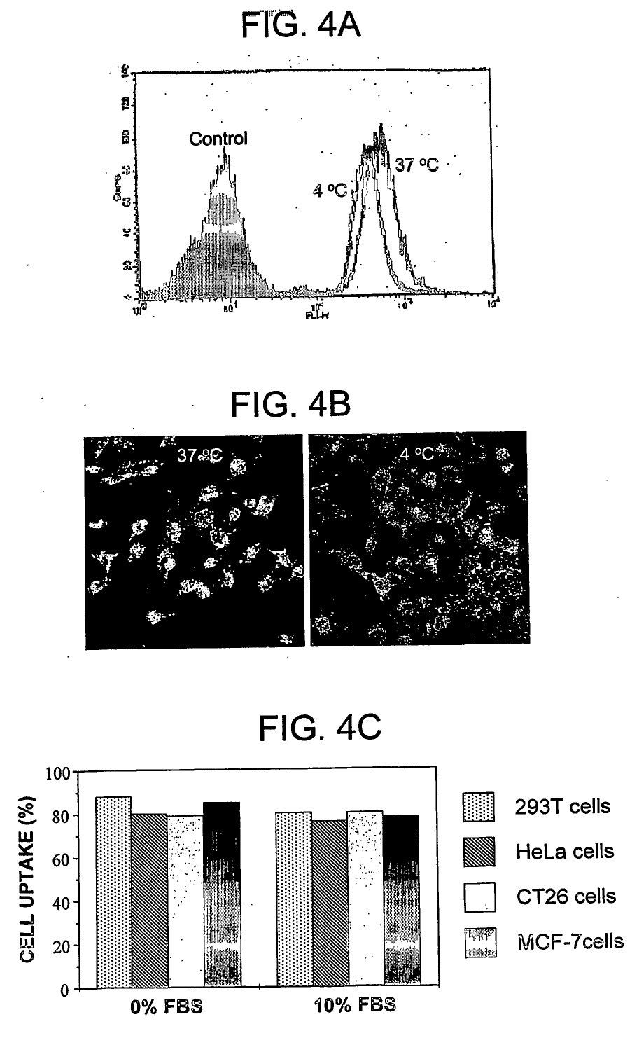

After 30 minutes incubation under standard cell culture conditions, all of the studied LMWP peptides displayed efficient cell uptake (FIG. 3A). Uptake of peptides increased with increasing the arginine content in these peptide (FIG. 3D). FACS analysis of cell uptake confirmed this effect of arginine content on cell transduction, as cells transduced with TDSP5 exhibited the highest fluorescence intensity (FIG. 3B). Similar results were obtained using the other cell line types including HeLa, CT26, and human MCF-7 cells. Cell uptake kinetics for LMWP and TAT were similar (FIGS. 1A-1D), indicating that LMWP possesses a similar cell-penetration capability as TAT.

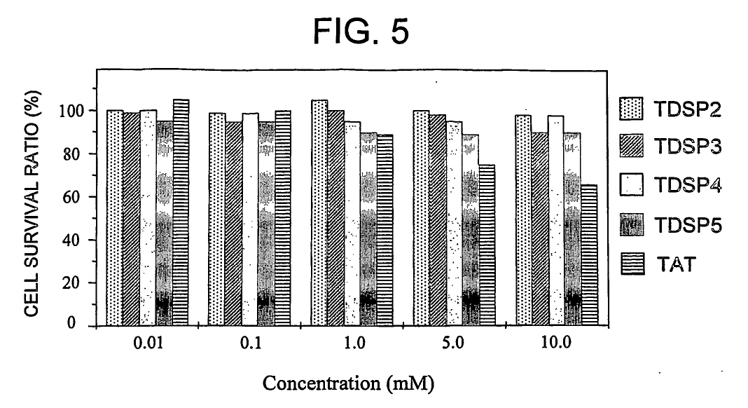

To examine if translocation of LMWP would follow a similar mechanism as that of TAT, cellular uptake experiments were conducted by incubating cells for 30 minutes at both 4° C. and 37° C. before applying the fluorescein-labeled TDSP4 or TDSP5. As shown by the FACS data (FIG. 4A) and supported by the observations of cellular fluorescence using confocal microscopy, the degree of translocation of TDSP5 at these two temperatures was almost identical. This finding indicated that, similar to TAT and other PTDs, internalization of TDSP5 was energy independent and did not follow the typical endocytosis pathway advocated for cellular uptake of ordinary small peptides (Schwarze et al., 1999; Suzuki et al., 2002).

To evaluate the effect of serum, identical experiments were conducted in cell culture medium containing 10% fetal bovine serum. The efficiency of TDSP5 uptake was not affected by the presence of serum, supporting the feasibility of in vivo applications (FIG. 4C).

### Example 4

### Cytotoxicity of LMWP Peptides

Cytotoxicity of the LMWP peptides and TDSP5-gelonin conjugates were examined using the cell lines described in Example 3. In brief, cells grown to 75% confluency were incubated with various concentrations of LMWP peptides, TAT peptide, TDSP5-gelonin conjugate, or TAT-gelonin conjugates. Cell proliferation was measured over 3 days, and cytotoxicity was determined using a colorimetric assay, which was performed by removing the cell culture medium and replacing it with PBS containing 5 mg/ml of 3-(4,5-dimethylthiazol-2-yl)-2,5-diphenyltetrazolium bromide (MTT). The absorbance of the cell culture medium following MTT addition was measured at 540 nm, and the survival ratio was determined by the ratio of the absorbance of sample-treated cells to that of control cells.

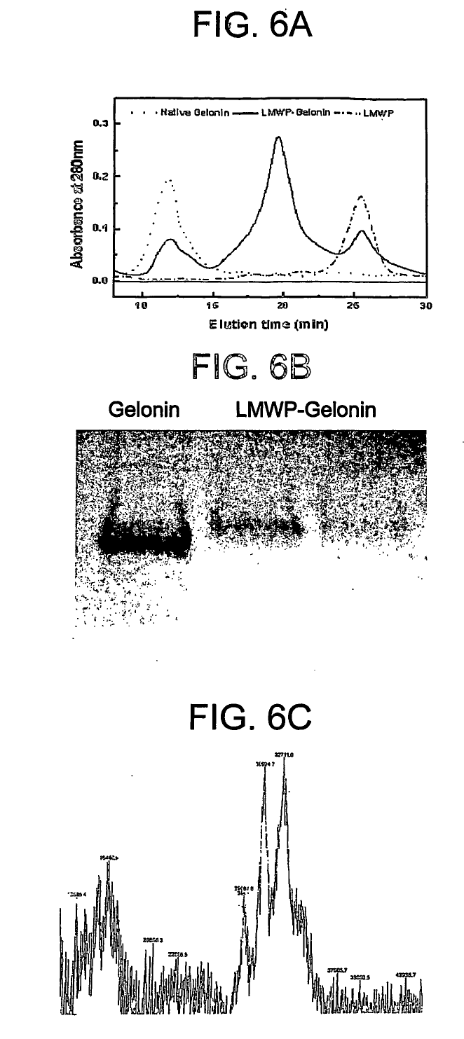

Little or no cellular cytotoxicity (i.e., <10% reduction in cell viability) was observed following incubation with LMWP peptides (concentrations up to 10 mM) (FIG. 5). In contrast, a significant decrease of cell viability (30-40%) was observed when cells were treated with TAT (5.0 mM-10.0 mM) (FIG. 5).

### Example 5

### Preparation of LMWP-Gelonin Conjugate

LMWP-gelonin conjugates were prepared essentially according to the procedures described by Carlsson et al. (1978) Biochem J 173:723-37. Briefly, 5 mg of TDSP5 was first reacted with N-succinimidyl 3-(2-pyridyldithio) propionate (SPDP) for 2 hours at room temperature, and was then thiolated with dithiothreitol (DTT) to create the —SH group at the N-terminal of TDSP5. Gelonin was activated with 3-(2-pyridyldithio) propionyl hydrazide (PDPH) and then reacted with the thiolated TDSP5. The reaction mixture was incubated overnight at 4° C. at pH 8.5.

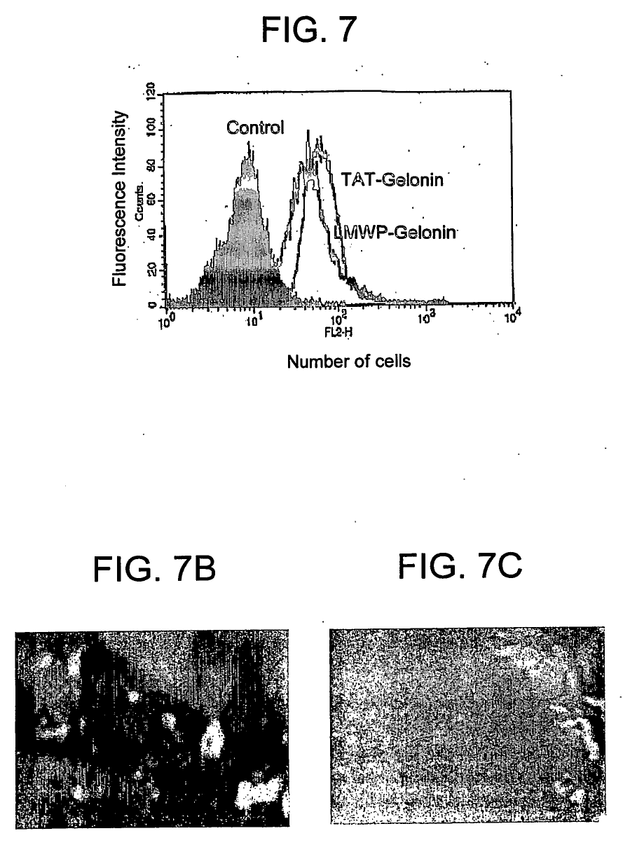

The final TDSP5-gelonin conjugate was purified using heparin affinity chromatography and then characterized for molecular weight and conjugation ratio using the SDS-PAGE. The conjugate was eluted at an ionic strength in-between those observed for gelonin and TDSP5, indicating the successful attachment of the heparin-binding TDSP5 to gelonin (FIG. 6A). Since TDSP5 possessed only one single —NH₂ group at the N-terminal end for conjugation with gelonin, a 1:1 molar ratio between TDSP5 and gelonin in the conjugate was expected. Results from SDS-PAGE (FIG. 6B) and MALDI-MS analysis (FIG. 6C) both revealed a 1:1 ratio of TDSP5:gelonin in the conjugate. TAT-gelonin was similarly prepared for use in control experiments.

### Example 6

### Cell Transduction of LMWP-Gelonin

Gelonin is among a class of hydrophilic, macromolecular drugs whose therapeutic functions are limited by poor cellular uptake. Indeed, despite its potent inhibitory action on protein synthesis, native gelonin has effected only low cytotoxicity towards tumors due to its inability to penetrate through the cell membrane. See Wu (1997) Br J Cancer 75: 1347-55. LMWP-gelonin conjugates were prepared as described in Example 5.

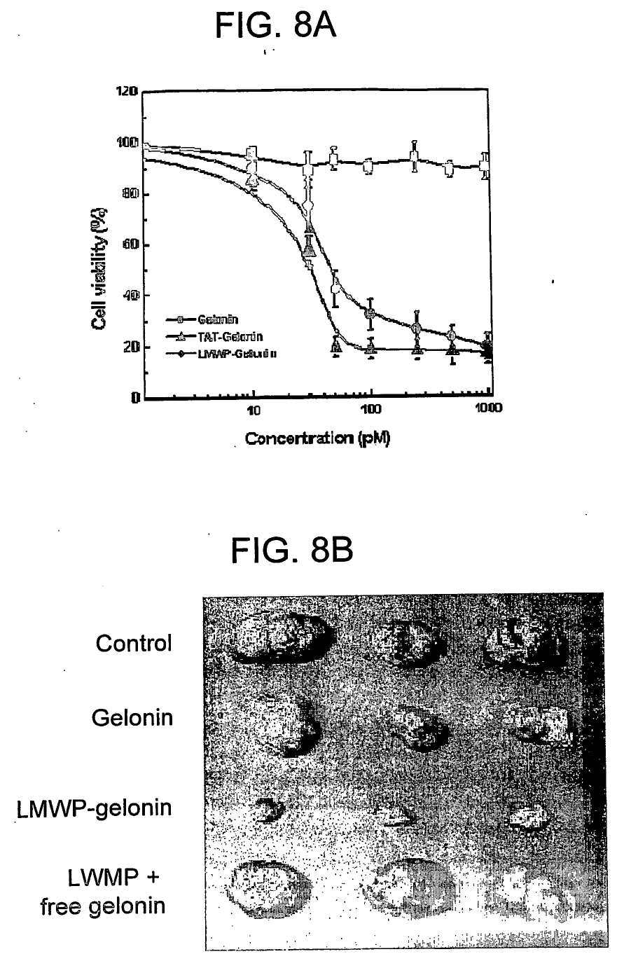

Cell transduction assays were preformed essentially as described in Example 3, with the exception that cells were incubated for 1 hour with the FITC-labeled peptide-gelonin conjugates. LMWP(TDSP5) and TAT showed a similar ability to translocate gelonin into cells (FIG. 7A). For both conjugates, rhodamine labels were clearly detected after 1 hour of incubation of these peptides with CT-26 cells. Data from confocal microscopic studies showed cytoplasmic localization of the TDSP5-gelonin conjugates.

Penetration of LMWP-gelonin conjugates was also tested in vivo using a mouse model of colon cancer. Tumors were established by subcutaneous injection of CT26 cells. Rhodamine-labeled LMWP-gelonin or rhodamine-labeled free gelonin was administered to tumor-bearing mice. As shown in FIG. 7B, rhodamin-labeled LMWP-gelonin accumulated in tumor cells. In contrast, rhodamine-labeled free gelonin displayed little or no accumulation in tumor cells (FIG. 7C). These results suggested that the antitumor effect of LMWP-gelonin was attributable, at least in part, to the enhanced distribution of the conjugates in tumor cells.

### Example 7

### Cytotoxicity and Anti-Tumor Activity of LMWP-Gelonin Conjugates

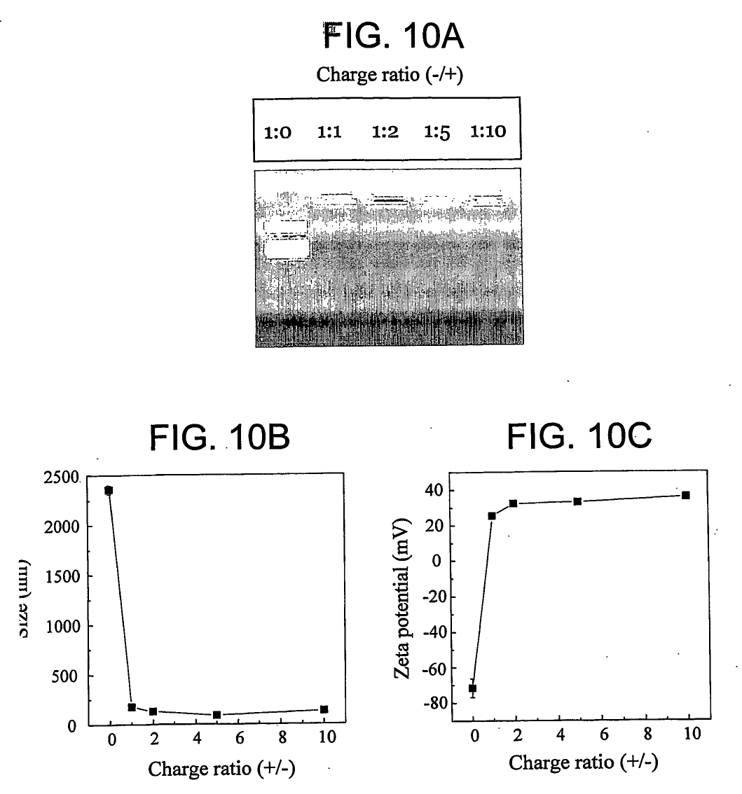

Cytotoxicity of CT26 cells when exposed to gelonin, TDSP5-gelonin, and TAT-gelonin was examined using a MTT assay as described in Example 4. As expected, gelonin itself did not cause any detectable inhibition on cell growth (FIG. 8A). In addition, co-administration of TDSP5 with gelonin did not yield any effect on the cytotoxicity, indicating that gelonin was unable to cross the cell membrane. In contrast, both the TAT-gelonin and TDSP5-gelonin conjugates were found to be highly cytotoxic, with IC₅₀ values (measured by the MTT assay) being about 10⁻⁸M (FIG. 8A). These results show that covalent linkage of the cell-impermeable gelonin to TDSP5 would enable a successful intracellular delivery of this protein toxin into cancer cells for possible therapeutic use.

The cytotoxicity of LMWP-gelonin conjugates was also tested in vivo using a mouse model of colon cancer. Tumors were established by subcutaneous injection of CT26 cells. Tumor-bearing mice were treated with PBS (Control); 100 μg of gelonin; 10 μg of LMWP and free gelonin mixture; or 110 μg of LMWP-gelonin (equivalent to 100 μg gelonin). Drugs were administered by intratumoral injection 3 weeks after tumor cell implantation or when tumors reached the size about 100 mm³. Thirty days (4 weeks) after initial treatment, mice were sacrificed and their tumors were excised, weighed and photographed (FIG. 8B). The tumors continued to grow in control mice injected with PBS solution (average tumor mass 3.16±0.65 g). Mice treated with free gelonin did not display regression on tumor growth, indicating that unconjugated gelonin could not penetrate the tumor (average tumor mass 2.63±0.5 g; p<0.05 when compared to control). In contrast, mice treated with the LMWP-gelonin conjugate displayed significant regression of tumor growth (average tumor mass 0.33±0.12 g). Addition of free LMWP to the gelonin solution did not elicit any effect on tumor regression (average tumor mass 2.74±0.68 g). These results demonstrate that LMWP-gelonin elicted anti-tumor activity.

### Example 8

### Preparation of pDNA/LMWP(TDSP5) Complexes

The pSV-β-galactosidase plasmid (Promega of Madison, Wis.) was amplified in Escherichia coli strain DH5α (Gibco-BRL of Gaithersburg, Md.) and purified by using QIAGEN® plasmid Maxi Kits (Qiagen of Valencia, Calif.). Purity of the plasmid DNA was confirmed by the OD₂₆₀/OD₂₈₀ ratio and the intensity of corresponding DNA fragments in gel electrophoresis following treatment of the plasmid DNA with a restriction enzyme. The concentration of the plasmid DNA was determined using the ratio that OD₂₆₀ value of 1 was equivalent to 50 μg of DNA. The plasmid DNA was stored at −20° C. until use.

pDNA/LMWP(TDSP5) complexes were prepared by mixing various amounts of LMWP (increasing from 1 μg to 20 μg in 10 μl water) with plasmid (1 μg in 10 μl water). For complex formation, the solution was left undisturbed for 30 minutes at 20° C. By mixing pDNA with various amounts of LMWP, pDNA/LMWP complexes comprising different charge ratios (−/+) ranging from 1:1 to 1:20 were obtained. Formation of pDNA/LMWP complexes was monitored using a gel retardation assay.

FITC-labeled pDNA/LMWP and pDNA/TAT complexes were prepared by mixing LMWP or TAT with FITC-labeled pDNA (pGENEGRIP™ plasmid and labeling kit, available from Gene Therapy Systems of San Diego, Calif.) encoding β-galactosidase at a charge ratio (−/+)=1:10. The solutions were incubated at 20° C. for 30 minutes to allow complex formation.

The size and zeta potential of pDNA/LMWP complexes were also examined using a Zeta-PALS zetameter (Brookhaven Instruments Corporation of Holtsville, N.Y.). All such experiments were carried out at 25° C., pH 7.0, 677 nm wavelength, and a constant angle of 15°. The particle size was presented as the effective mean diameter.

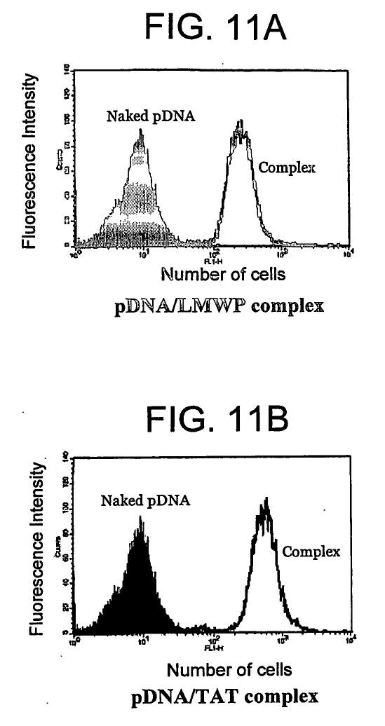

The charge ratio (−/+) used in the preparation of the pDNA/LMWP complex was controlled at 1:2. After complex formation, DNase I (50 units, available from Gibco BRL of Gaithersburg, Md.) was added to the complex suspension, and the solution was incubated at 37° C. for 60 minutes. Naked pDNA was used as a control. At time intervals of 0, 10, 20, 40, 60, 80 minutes incubation, 50 μl of the complex suspension were withdrawn, mixed with 75 μl of the stop solution (4M ammonium acetate, 20 mM EDTA, and 2 mg/ml glycogen), and then placed on ice. The pDNA was dissociated from LMWP by adding 37 μl 1.0% SDS to the complex suspension and then heating the mixture at 65° C. for overnight. The pDNA was extracted and precipitated by treating the solution mixture with phenol/chloroform and ethanol several times. The precipitated DNA pellet was then dissolved in 10 μl of TE buffer and resolved using 1.0% agarose gel electrophoresis. Results of a DNase I protection assay, which was performed using the pDNA/LMWP(TDSP5) complex, showed that complexed pDNA was protected from degradation (FIG. 9).

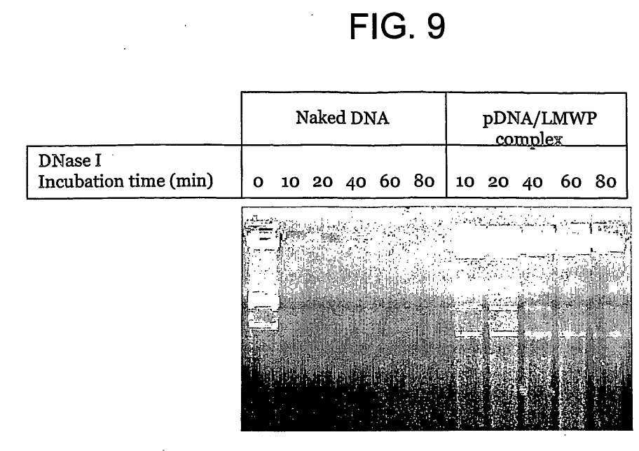

The migration of pDNA/LMWP complexes (1:1 charge ratio (−/+) between pDNA and LMWP(TDSP5)) was retarded (FIG. 10A), indicating that LMWP was able to effectively condense plasmid DNA into a complex at a charge ratio of 1:1 (−/+). These findings are in good agreement with those observed by other investigators, indicating that LMWP formed a condensed complex with pDNA as effectively as any of the currently used cationic polymeric gene carriers. See e.g., Midoux et al. (1999) Bioconjug Chem 10:406-11 and Zauner et al. (1996) Biotechniques 20:905-13.

While the naked DNA has a size of 2300 nm, complexation of pDNA and LMWP(TDSP5) produced particles with significantly reduced size, about 120 nm at a charge ratio (−/+) 1:2 (FIG. 10B). In addition, surface charge of the particle increased significantly after the formation of the plasmid DNA/LMWP(TDSP5) complex. Unlike the naked plasmid, which DNA showed a surface charge of −70 mV, pDNA/LMWP(TDSP5) complexes showed a surface charge positive of 30 mV (FIG. 9C). It has been previously documented that particles with a size below 200 nm and a surface charge of about 20 mV to −30 mV can rapidly enter cells in vitro (Midoux et al. (1999) Bioconjug Chem 10:406-11). Thus, these results are consistent with membrane-translocating ability of pDNA/LMWP(TDSP5).

### Example 9

### Cell Transduction of pDNA/LMWP(TDSP5) Complexes

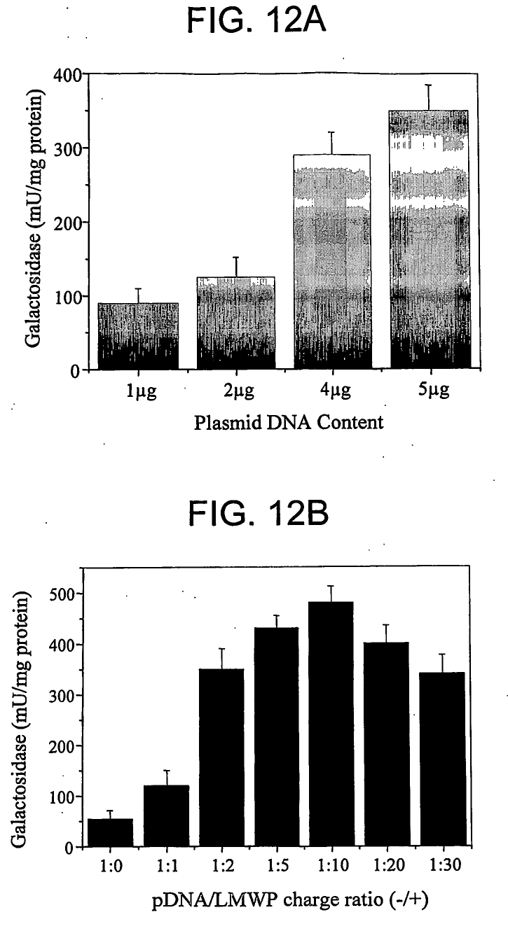

Cell transfection assays were performed using FITC-labeled pDNA/LMWP(TDSP5) complexes essentially as described in Example 4. The efficiency of transfection to 293T cells in the presence of pDNA/LMWP(TDSP5) was markedly higher than that of naked DNA alone (FIGS. 11A, B).

Cell transduction was also assessed using a histochemical assay as follows. 293T cells were seeded at a density of 2×10⁶ cells/dish in 35-mm culture dishes, and incubated for 24 hours before the addition of transfection complexes. Transfection mixtures were prepared separately for LMWP and PEI; whereas PEI was served as a control. pDNA/LMWP (or pDNA/PEI) complexes were prepared by mixing 10 μg of pSV-β-galactosidase and various amounts of LMWP (or PEI) in 500 μl of serum free DMEM medium, followed by incubating the mixtures for 30 minutes at room temperature. The molecular weight of PEI for transfection and cytotoxicity studies was either 2000 or 25000 Daltons. 500 μl of LMWP/DNA complex was then added to each well, and the cells were incubated for 4 hours at 37° C. in a 5% CO₂ incubator. After 6-hour exposure, the transfection mixtures were replaced with 2 ml of fresh DMEM medium containing 10% FBS and 100 units/ml penicillin-streptomycin mixture. Cells were incubated for an additional 2 days at 37° C. before analysis of β-galactosidase activity.

To perform the β-galactosidase assay, cells were washed, lysed with 300 μl of lysis reagent (25 mM glycylglycine, 15 mM MgSO₄, 4 mM EDTA, 1% TRITON-X®100 detergent, 1 mM DTT, 1 mM PMSF), and centrifuged at 13,000 rpm for 5 minutes. The activity of β-galactosidase in the supernatants was then measured using an ONPG assay essentially as described by Lampela et al. (2002) J Gene Med 4:205-14. The ONPG assay was based on cleavage of the β-bond of ONPG by β-galactosidase, resulting in the production of a yellow o-nitrophenol molecule. The reaction was quenched using 1M Na₂CO₃. Samples were analyzed by measuring the absorbance at 420 nm with a Bio-Rad microplate reader (BioRad Laboratories of Hercules, Calif.). In addition, protein concentration in the cell supernatants was measured using a BCA (bicinchoninic acid) protein assay kit (Biorad Laboratories of Hercules, Calif.).

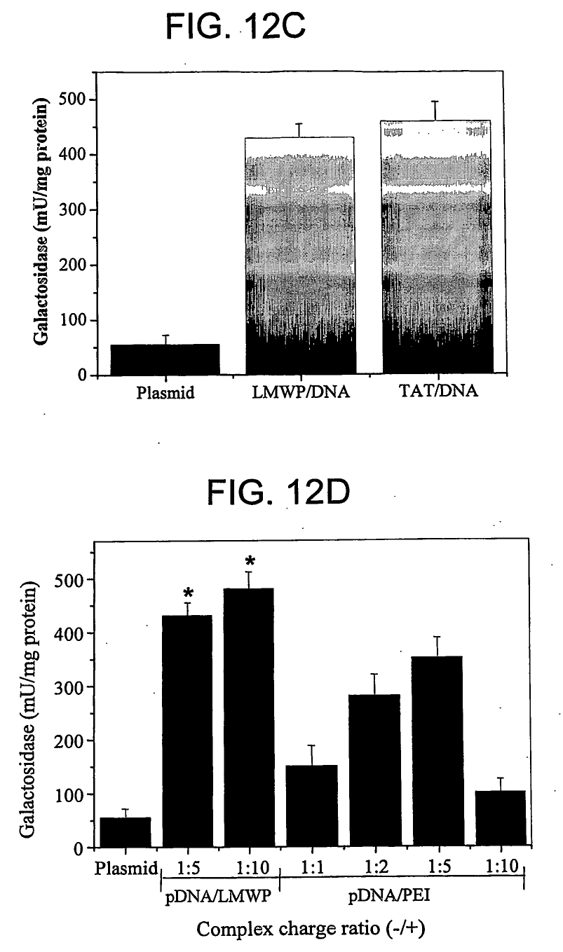

At a charge ratio of 1:2 (−/+), the transfection efficiency increased with increasing the plasmid DNA content (FIG. 12A). When plasmid content was fixed at 5 μg, transfection efficiency was increased to a maximum when the charge ratio of the complex was raised to 1:10 (−/+) (FIG. 12B). Further increase of the charge ratio of the complex above 1:20 (−/+) resulted in no more increase of transfection efficiency (FIG. 12B). The transfection efficiency decreased when the charge ratio of the complex was increased above 1:20 (−/+)(FIG. 12B). This reduced transfection efficiency at the charge ratio above 1:20 (−/+) could be due to an aggregation of particles. The transfection efficiency of LMWP(TDSP5)/DNA was similar to TAT/DNA (FIG. 12C).

To further compare the transfection efficiency, experiments were conducted using polyethylene imine (PEI) with similar molecular weight to LMWP as the DNA carrier. PEI is a water soluble and cationic gene carrier and has been known to be a potent carrier of pDNA internalization into the cells. See Wightman et al. (2001) J Gene Med 3: 362-72; Abdallah et al. (1996) Hum Gene Ther 7: 1947-54. Various charge ratios of pDNA/PEI complexes were transfected into 293T cells. The pDNA/PEI complex yielded the highest transfection efficiency when prepared at a charge ratio of 1:5 (FIG. 12D). At this same charge ratio, pDNA/LMWP(TDSP5) mediated a higher transfection efficiency (i.e. 26% increased transfection) than PEI (FIG. 12D). Similarly, at a charge ratio (−/+) of 1:10, which is the ratio for achieving the maximum transfection efficiency for pDNA/LMWP(TDSP5), transfection efficiency of pDNA/LMWP(TDSP5) was also markedly higher than that of pDNA/PEI (FIG. 12D).

### Example 10

### Cyotoxicity of pDNA/LMWP(TDSP5) Complexes

Cytotoxicity of LMWP and pDNA/LMWP complexes was evaluated using a MTT assay. In general, the 293T cells were seeded at a density of 1.0×10⁴ cells/well in the 96-well flat-bottomed microassay plate (Falcon Co. of Becton Dickinson, Franklin Lakes, N.J.) and incubated for 24 hours. The LMWP(TDSP5) or pDNA/LMWP(TDSP5) complex solution was then added and the mixture was incubated for another 4 hours at 37° C. Parallel experiments were conducted using PEI and pDNA/PEI complex as controls. At the end of the transfection experiment, the medium was replaced with 200 μl of fresh DMEM medium without serum, and 125 μl of 2 mg/ml MTT solution in PBS were then added. After incubation for an additional 4 hours at 37° C., the MTT-containing medium was removed, 200 μl of DMSO were added to dissolve the formazan crystal formed by live cells. Absorbance was measured at 570 nm. Cell viability (%) was calculated according to the following equation:
Cell viability (%)=(OD₅₇₀₍ₛₐₘₚₗₑ₎)/OD_{570(control)})×100
where the OD₅₇₀₍ₛₐₘₚₗₑ₎ and _{OD570(control)} represented measurements from wells treated with LMWP(TDSP5)/DNA and PBS buffer, respectively.

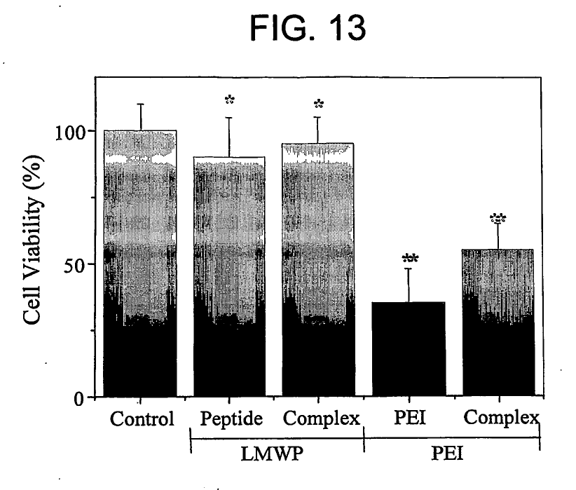

Cells treated with LMWP showed similar viability in comparison to untreated cells (FIG. 13). When pDNA/LMWP(TDSP5) complexes (5 μg of pDNA, charge ratio 1:10) were added to 293T cells, negligible cytotoxicity was observed (FIG. 13). In contrast, cells treated with PEI introduced cytotoxicity to the cells by reducing the cell viability to about 35% (FIG. 13). Treatment of cells with pDNA/PEI also resulted in substantial reduction of viability. Consistent with previous reports (Morgan, 1990), PEI showed greater toxicity when administered without bound DNA.

While the present invention has been described in connection with what is presently considered to be practical and preferred embodiments, it is understood that the present invention is not to be limited or restricted to the disclosed embodiments but, on the contrary, is intended to cover various modifications and equivalent arrangements included within the scope of the appended claims. Thus, it is to be understood that variations in the described invention will be obvious to those skilled in the art without departing from the novel and non-obvious aspects of the present invention, and such variations are intended to come within the scope of the claims below.

What is claimed is:

1. A composition for transport across a biological membrane comprising a membrane-translocating LMWP peptide and a cargo molecule, wherein the LMWP peptide is conjugated to, complexed with, fused to, or otherwise in association with the cargo molecule.

2. The composition of claim 1, wherein the membrane-translocating LMWP peptide comprises any one of SEQ ID NOs: 1-4.

3. The composition of claim 2, wherein the membrane-translocating LMWP peptide comprises SEQ ID NO: 1.

4. The composition of claim 2, wherein the membrane-translocating LMWP peptide comprises SEQ ID NO: 2.

5. The composition of claim 2, wherein the membrane-translocating LMWP peptide comprises SEQ ID NO: 3.

6. The composition of claim 2, wherein the membrane-translocating LMWP peptide comprises SEQ ID NO: 4.

7. The composition of claim 1, wherein the membrane-translocating LMWP peptide comprises a purified thermolysin-digested protamine peptide.

8. The composition of claim 1, wherein the cargo molecule is a therapeutic agent, a diagnostic agent, a binding agent, or a heterologous agent.

9. The composition of claim 8, wherein the therapeutic agent is a cytotoxin.

10. The composition of claim 9, wherein the cytotoxin is a protein synthesis inhibitor.

11. The composition of claim 10, wherein the protein synthesis inhibitor is gelonin.

12. The composition of claim 8, wherein the cargo molecule is a diagnostic agent comprising a radionuclide, a metal ion, gas microbubbles, a fluorophore, an epitope, and a radioactive label.

13. The composition of claim 12, wherein the diagnostic agent is a fluorophore.

14. The composition of claim 1, wherein the cargo molecule is a peptide, a polypeptide, a nucleic acid, a small molecule, a polymeric conjugate, an antibody, a peptide nucleic acid, a carbohydrate, a vitamin, a hormone, an odorant, a pheromone, a toxin, or combination thereof.

15. The composition of claim 14, wherein the cargo molecule is a nucleic acid.

16. The composition of claim 15, wherein the nucleic acid is a plasmid.

17. The composition of claim 15, wherein the nucleic acid is complexed with the LMWP peptide via an ionic interaction.

18. The composition of claim 15, wherein the complexed nucleic acid is condensed.

19. The composition of claim 14, wherein the cargo molecule is a protein.

20. The composition of claim 19, wherein the protein is gelonin.

21. The composition of claim 1, further comprising a pharmaceutically acceptable carrier.

22. A pharmaceutical composition for drug delivery comprising:
(a) a composition for transport across a biological membrane comprising a membrane-translocating LMWP peptide and a drug, wherein the membrane-translocating LMWP peptide is conjugated to, complexed with, or fused to the therapeutic cargo molecule; and
(b) a pharmaceutically acceptable carrier.

23. The pharmaceutical composition of claim 22, wherein the LMWP peptide comprises any one of SEQ ID NOs: 1-4.

24. The pharmaceutical composition of claim 23, wherein the LMWP peptide comprises SEQ ID NO: 1.

25. The pharmaceutical composition of claim 23, wherein the LMWP peptide comprises SEQ ID NO: 2.

26. The pharmaceutical composition of claim 23, wherein the LMWP peptide comprises SEQ ID NO: 3.

27. The pharmaceutical composition of claim 23, wherein the LMWP peptide comprises SEQ ID NO: 4.

28. The pharmaceutical composition of claim 22, wherein the LMWP peptide comprises a purified thermolysin-digested protamine peptide.

29. The pharmaceutical composition of claim 22, wherein the drug is selected from the group consisting of a therapeutic agent, a diagnostic agent, a binding agent, and a heterologous agent.

30. The pharmaceutical composition of claim 29, wherein the therapeutic agent is a cytotoxin.

31. The pharmaceutical composition of claim 30, wherein the cytotoxin is a protein synthesis inhibitor.

32. The pharmaceutical composition of claim 31, wherein the protein synthesis inhibitor is gelonin.

33. The pharmaceutical composition of claim 29, wherein the diagnostic agent comprises a radionuclide, a metal ion, gas microbubbles, a fluorophore, an epitope, and a radioactive label.

34. The pharmaceutical composition of claim 33, wherein the diagnostic agent is a fluorophore.

35. The pharmaceutical composition of claim 22, wherein the drug is selected from the group consisting of a peptide, a polypeptide, a nucleic acid, a small molecule, an antibody, a peptide nucleic acid, a carbohydrate, a vitamin, a hormone, an odorant, a pheromone, a toxin, and combinations thereof.

36. The pharmaceutical composition of claim 35, wherein the drug is a nucleic acid.

37. The pharmaceutical composition of claim 36, wherein the nucleic acid is a plasmid.

38. The pharmaceutical composition of claim 36, wherein the nucleic acid is complexed with the LMWP peptide via an ionic interaction.

39. The pharmaceutical composition of claim 36, wherein the complexed nucleic acid is condensed.

40. The pharmaceutical composition of claim 35, wherein the drug is a protein.

41. The pharmaceutical composition of claim 40, wherein the protein is gelonin.

42. A method for transporting or enhancing the transport of a cargo molecule across a biological membrane, the method comprising contacting a biological membrane with a composition comprising a membrane-translocating LMWP peptide and a cargo molecule, whereby the cargo molecule is transported across a biological membrane.

43. The method of claim 42, wherein the biological membrane comprises a cell membrane or an intracellular membrane.

44. The method of claim 43, wherein the intracellular membrane is a nuclear membrane.

45. The method of claim 43, wherein the biological membrane is a eukaryotic cell membrane or a prokaryotic cell membrane.

46. The method of claim 45, wherein the eukaryotic cell is a mammalian cell.

47. The method of claim 46, wherein the mammalian cell is a human cell.

48. The method of claim 45, wherein the prokaryotic cell is a bacterial cell.

49. The method of claim 48, wherein the bacterial cell is part of a bacterial biofilm layer.

50. The method of claim 42, wherein the biological membrane is in vitro.

51. The method of claim 50, wherein the in vitro biological membrane is ex vivo.

52. The method of claim 42, wherein the biological membrane is in vivo.

53. The method of claim 42, wherein the membrane-translocating LMWP peptide comprises any one of SEQ ID NOs: 1-4.

54. The method of claim 53, wherein the membrane-translocating LMWP peptide comprises SEQ ID NO: 1.

55. The method of claim 53, wherein the membrane-translocating LMWP peptide comprises SEQ ID NO: 2.

56. The method of claim 53, wherein the membrane-translocating LMWP peptide comprises SEQ ID NO: 3.

57. The method of claim 42, wherein the membrane-translocating LMWP peptide comprises a purified thermolysin-digested protamine peptide.

58. The method of claim 42, wherein the cargo molecule is a therapeutic agent, a diagnostic agent, a binding agent, or a heterologous agent.

59. The method of claim 58, wherein the therapeutic agent is a cytotoxin.

60. The method of claim 59, wherein the cytotoxin is a protein synthesis inhibitor.

61. The method of claim 60, wherein the protein synthesis inhibitor is gelonin.

62. The method of claim 58, wherein the diagnostic agent comprises a radionuclide, a metal ion, gas microbubbles, a fluorophore, an epitope, or a radioactive label.

63. The method of claim 62, wherein the diagnostic agent is a fluorophore.

64. The method of claim 62, further comprising detecting the diagnostic agent.

65. The method of claim 42, wherein the cargo molecule is a peptide, a polypeptide, a nucleic acid, a small molecule, a polymeric conjugate, an antibody, a peptide nucleic acid, a carbohydrate, a vitamin, a hormone, an odorant, a pheromone, a toxin, or combination thereof.

66. The method of claim 65, wherein the cargo molecule is a nucleic acid.

67. The method of claim 66, wherein the nucleic acid is a plasmid.

68. The method of claim 66, wherein the nucleic acid is complexed with the LMWP peptide via an ionic interaction.

69. The method of claim 66, wherein the complexed nucleic acid is condensed.

70. The method of claim 67, wherein the cargo molecule is a protein.

71. The method of claim 70, wherein the protein is gelonin.

72. A method for drug delivery to a subject, the method comprising administering to a subject a composition for transport across a biological membrane, wherein the composition comprises a membrane-translocating LMWP peptide, a drug, and a pharmaceutically acceptable carrier; and whereby the drug is delivered to cells of the subject.

73. The method of claim 72, wherein the subject is a mammal.

74. The method of claim 73, wherein the mammal is a human.

75. The method of claim 72, wherein the membrane-translocating LMWP peptide comprises any one of SEQ ID NOs: 1-4.

76. The method of claim 75, wherein the membrane-translocating LMWP peptide comprises SEQ ID NO: 1.

77. The method of claim 75, wherein the membrane-translocating LMWP peptide comprises SEQ ID NO: 2.

78. The method of claim 75, wherein the membrane-translocating LMWP peptide comprises SEQ ID NO: 3.

79. The method of claim 75, wherein the membrane-translocating LMWP peptide comprises SEQ ID NO: 4.

80. The method of claim 72, wherein the membrane-translocating LMWP peptide comprises a purified thermolysin-digested protamine peptide.

81. The method of claim 72, wherein the drug is selected from the group consisting of a therapeutic agent, a diagnostic agent, a binding agent, and a heterologous agent.

82. The method of claim 81, wherein the therapeutic agent is a cytotoxin.

83. The method of claim 82, wherein the cytotoxin is a protein synthesis inhibitor.

84. The method of claim 83, wherein the protein synthesis inhibitor is gelonin.

85. The method of claim 81, wherein the diagnostic agent comprises a detectable label selected from the group consisting of a radionuclide, a metal ion, gas microbubbles, a fluorophore, and an epitope.

86. The method of claim 85, wherein the diagnostic agent is a fluorophore.

87. The method of claim 72, wherein the drug is selected from the group consisting of a peptide, a polypeptide, a nucleic acid, a small molecule, a polymeric conjugate, an antibody, a peptide nucleic acid, a carbohydrate, a vitamin, a hormone, an odorant, a pheromone, a toxin, and combinations thereof.

88. The method of claim 87, wherein the drug is a nucleic acid.

89. The method of claim 88, wherein the nucleic acid is a plasmid.

90. The method of claim 87, wherein the nucleic acid is complexed with the LMWP peptide via an ionic interaction.

91. The method of claim 87, wherein the complexed nucleic acid is condensed.

92. The method of claim 72, wherein the drug is a protein.

93. The method of claim 92, wherein the protein is gelonin.


SEQUENCE LISTING (4 sequences)

SEQ ID NO: 1 - protein, 6 aa, Salminus sp.
PRRRRR

SEQ ID NO: 2 - protein, 10 aa, Salminus sp.
PRRRRSSRRP

SEQ ID NO: 3 - protein, 13 aa, Salminus sp.
ASRRRRRGGRRRR

SEQ ID NO: 4 - protein, 14 aa, Salminus sp.
VSRRRRRRGGRRRR